(19) United States
(12) Patent Application Publication
(10) Pub. No.: US 2006/0052384 A1
(43) Pub. Date: Mar. 9, 2006

(54) Aryl piperidine derivatives as inducers of ldl-receptor expression for the treatment of hypercholesterolemia

(76) Inventors: Anne Marie Jeanne Bouillot, Les Ulis (FR); Bernard Andre Dumaitre, Les Ulis (FR)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/520,799

(22) PCT Filed: Jul. 11, 2003
(86) PCT No.: PCT/EP03/07612

§ 371 Date: Jan. 10, 2005

(30) Foreign Application Priority Data
Jul. 12, 2002 (GB) 0216224.6

(51) Int. Cl. A61K 31/506; A61K 31/501; A61K 31/454; C07D 403/02; C07D 401/02

(52) U.S. Cl. 514/252.03; 514/256; 514/318; 514/319; 514/323; 514/326; 544/238; 544/333; 546/194; 546/201; 546/205; 546/207; 546/210; 546/211

(57) ABSTRACT

This invention relates to novel compounds which up-regulate LDL receptor (LDL-r) expression and to processes for their preparation, pharmaceutical compositions containing them and their medical use. More particularly, this invention relates to novel aromatic piperidines and their use in therapy.

This invention relates to novel compounds which up-regulate LDL receptor (LDL-r) expression and to processes for their preparation, pharmaceutical compositions containing them and their medical use. More particularly, this invention relates to novel aromatic piperidines and their use in therapy.

Epidemiological studies have clearly demonstrated the correlation between reduction in plasmatic LDL cholesterol and the benefit on cardiovascular events including mortality. LDL cholesterol is eliminated from plasma by specific binding to LDL-r expressed by the liver. Regulation of LDL-r expression occurs in the liver and is mainly dependent on intracellular cholesterol concentration. Increasing free cholesterol concentration leads to a reduced LDL-r expression through a mechanism involving transcriptional factors. Counteracting with this process is expected to up-regulate LDL-r expression in the liver and to increase the clearance of LDL cholesterol.

International Patent Application Number PCT/EP00/06668 concerns the novel use of the SREBP-cleavage activating protein (SCAP) in a screening method. Two compounds are disclosed, namely 4-(4-chloro-benzoylamino)-N-{4-[4-(2-ethoxy-4-ethyl-phenyl)-piperidin-1-yl]-butyl}-benzamide and 4-(4-benzoyl)-N-{4-[4-(4-isopropyl-2-methoxy-phenyl)-piperidin-1-yl]-butyl}-benzamide hydrochloride, which do not form part of the present invention.

Another publication, Bioorganic and Medicinal Chemistry Letters Vol. 5, 3, 219-222, 1995 discloses compounds having the general formula (A) 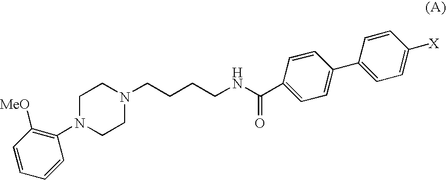
where X may be COMe, SO₂Me and NH₂, as having high affinity for the dopamine D₃ receptor and postulates their use in CNS disorders, particularly psychiatric illness. The compound of formula A where X is COMe is also disclosed in J. Pharmacol. Exp. Ther. 287; 1 1998 187-197 and Bioorganic and Medicinal Chemistry Letters Vol. 7, 15, 1995-1998, 1997, again as being useful in treating CNS disorders. It will be noted that the present invention differs from the compounds of formula (A) in use of a piperidine ring rather than a piperazine and in the utility disclosed.

Journal Of Medicinal Chemistry, Vol. 40, 6, 952-960, 1997 discloses compounds of formula (B) 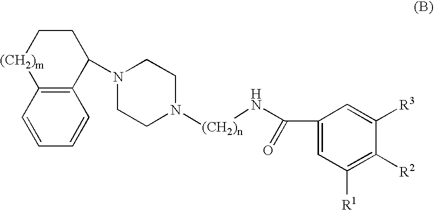
where m=0, 1 or 2; n=2 or 3; R¹ and R³═H or OMe and R² may be Ph, as selective 5-HT_{1A} receptor ligands having CNS activity. It will be noted that the examples of the present invention differ from those of formula (B) in use of a piperidine ring rather than a piperazine and in the utility disclosed.

International Patent Application Publication Number WO99/45925 discloses compounds of formula (C) 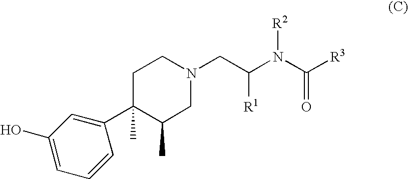
where R¹ may be hydrogen, R² may be hydrogen and R³ may be a group 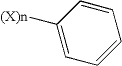
where X may be an aryl group and n may be 1. Specifically disclosed are compounds where the group COR³ is formed from 2- and 4-biphenyl carboxylic acid and R¹ and R² are methyl or hydrogen respectively. The utility of the compounds is as opioid receptor binding agents which may be useful as analgesics. The substitution on the 3- and 4-positions of the piperidine ring leave the compounds of this publication outside the scope of the present invention. Furthermore, the utility disclosed is different.

International Patent Application Publication Number WO98/37893 discloses compounds of formula (D) 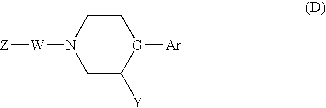
where Ar may represent an optionally substituted phenyl or naphthyl, G may be N or CH₂ (sic), W may be an optionally substituted alkylene, Y may be hydrogen and Z may represent a group R⁴CONR⁵, where R⁴ may be an optionally substituted phenyl and R₅ may be hydrogen. These compounds are described as being D2 receptor antagonists useful in the treatment of CNS disorders such as Parkinson's Disease. None of the compounds specifically disclosed fall within the scope of the present invention and the disclosed utlity is different.

International Patent Application Publication Number WO9402473 discloses compounds of formula (E)

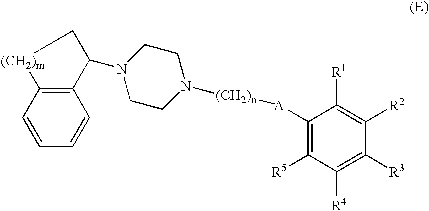

where A is —NHCO— or —CONH—; R₁—R₅ may be hydrogen or phenyl, m may be 1-3 and n may be 1-3. Specifically disclosed are the following compounds:

| No. | A | n | m | R¹ | R² | R³ | R⁴ | R⁵ |
|---|---|---|---|---|---|---|---|---|
| 5 | NHCO | 2 | 1 | H | H | Ph | H | H |
| 12 | NHCO | 2 | 2 | H | H | Ph | H | H |
| 19 | NHCO | 2 | 3 | H | H | Ph | H | H |

The compounds are described as 5HT-1A agonists having CNS activity and may be used as anti-depressants, anti-hypertensive, analgesics etc. It will be noted that the examples of the present invention differ from those of formula (E) in use of a piperidine ring rather than a piperazine and in the utility disclosed.

International Patent Application Publication Number WO99/45925 discloses compounds of formula (F) 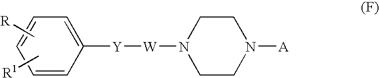
where A may represent a substituted phenyl group, W represents a linear or branched alkylene group having from 2 to 6 carbon atoms; Y may represent a group NHCO or CONH; and R may be a substituted phenyl group. Particularly disclosed is the compound G

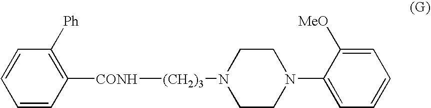

These compounds are described as being α1A-adrenergic receptors useful in the treatment of contractions of the prostate, urethra and lower urinary tract, without affecting blood pressure. It will be noted that the examples of the present invention differ from those of formula (G) in use of a piperidine ring rather than a piperazine and in the utility disclosed.

International Patent Application Publication Number WO98/35957 describes compounds of formula (H) 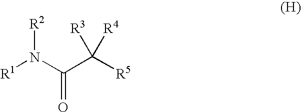
wherein R¹—R⁵ are each individually selected from the group of substituents including hydrogen, halogen, hydroxyl, thiol, lower alkyl, substituted lower alkyl, alkenyl, alkynyl, alkylalkenyl, alkylalkynyl, alkoxy, alkylthio, acyl, aryloxy, amino, amido, carboxyl, aryl, substituted aryl, heterocycle, heteroaryl, substituted heterocycle, heteroalkyl, cycloalkyl, substituted cycloalkyl, alkylcycloalkyl, alkylcycloheteroalkyl, nitro and cyano. Specifically disclosed compounds are those formed by the N-alkylation of a a substituted piperidine or piperazine with a group (J) 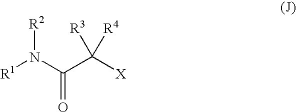
where X is a leaving group. None of the compounds specifically disclosed fall within the scope of the present invention and the invention is in no way suggested by the disclosure. The compounds are said to be of use as NPY Y5 receptor antagonists in the treatment of obesity, bulemia and related disorders and NPY Y5 receptor inhibition related disorders such as memory disorders, epilepsy, dyslipidemia and depression.

Journal Of Medicinal Chemistry, Vol. 31, 1968-1971, 1988 discloses certain aryl piperazines compounds, which fall outside the present invention, as 5HT-1a Serotonin Ligands as potential CNS agents. Specifically disclosed are compounds of formula (K) 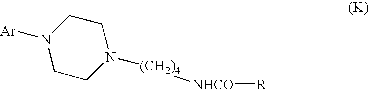
where Ar=Ph and R=Ph, Ar=2-methoxyphenyl and R=Ph and Ar=2-pyrimidyl and R=Ph.

Journal Of Medicinal Chemistry, Vol. 34, 2633-2638, 1991 discloses aryl piperazines having reduced α1 adrenergic affinity. Specifically disclosed is the compound (L) 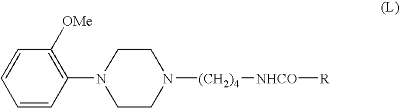
where R is 4-(BnO)-phenyl, which falls outside the scope of the present invention.

The present invention provides aryl piperidine derivatives which are particularly useful in treating cardiovascular disorders associated with elevated levels of circulating LDL-cholesterol.

According to a first aspect, the invention provides a compound of formula (I), a physiologically acceptable prodrug, salt or solvate thereof; 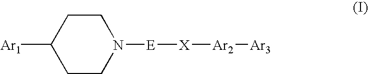
wherein
Ar₁ is:
(i) phenyl, naphthyl or phenyl fused by a C₃₋₈cycloalkyl; or
(ii) heterocyclyl selected from the list consisting of: monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated or aromatic, provided that at least one ring is aromatic;
   where Ar₁ is optionally substituted by 1-4 R¹ groups which may be the same or different;
Ar₂ is a phenyl group, a 5-6 membered heteroaromatic group or a bicyclic heteroaromatic group, each of which is optionally substituted by 1-4 groups independently selected from the list: C₁₋₄alkyl, halogen, hydroxy, C₁₋₄alkoxy, C₁₋₆acyl, C₁₋₆acyloxy, amino, C₁₋₄alkylamino, di-C₁₋₄alkylamino, —(CH₂)ₙOH, —(CH₂)ₙNRₓR_{y}, —O(CH₂)ₙO(CH₂)ₘOR^{a}, —O(CH₂)ₙC(O)NRₓR_{y}, —O(CH₂)ₙCN, C₂₋₅alkenyl, —O(CH₂)ₙCO₂R^{a}, —OSO₂(CH₂)ₚCH₃, —OSO₂NRₓR_{y} and —CO₂(CH₂)ₚCH₃;
Ar₃ is:
(i) phenyl, naphthyl or phenyl fused by a C₃₋₈cycloalkyl; or
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
   wherein Ar₃ is optionally substituted by 1-4 groups independently selected from the group consisting of: hydroxy, C₁₋₄alkyl, C₁₋₄alkoxy, C₂₋₄alkenyl, C₂₋₄alkenyloxy, C₁₋₄perfluoroalkoxy, C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, nitro, C₁₋₄perfluoroalkyl, C₁₋₄alkylcarbonyl, fluoroC₁₋₄alkylcarbonyl, C₁₋₄alkoxycarbonyl, aminocarbonyl, C₁₋₄alkylaminocarbonyl, di-C₁₋₄alkylaminocarbonyl, C₁₋₄alkylsulfonyl, C₁₋₄alkylaminosulfonyl, di-C₁₋₄alkylaminosulfonyl, C₁₋₄alkylsulfonyl and C₁₋₄alkylsulfoxy;
E is —C₁₋₆alkylene-;
X is —CONR^{a}— or —NR^{a}CO— (where the left hand side of the linkage is attached to E);
wherein
R¹ is halogen, C₁₋₄,alkoxy or C₁₋₄alkyl;
R^{a} is C₁₋₄alkyl or hydrogen;
Rₓ and R_{y} are independently hydrogen, C₁₋₄alkyl, hydroxy or C₁₋₄alkoxy, where Rₓ and R_{y} are not both hydroxy or both C₁₋₄alkoxy; or Rₓ and R_{y} together with the nitrogen to which they are attached form a 5-membered ring which ring is optionally substited by —O(CH₂)ₙC(O)NRₓR_{y}, —O(CH₂)ₙCN, —O(CH₂)ₙO(CH₂)ₘOR^{a}, —O(CH₂)ₙCO₂R^{a}, —OSO₂NRₓR_{y}, —OSO₂(CH₂)ₚCH₃, —(CH₂)ₙC(O)NRₓR_{y}, —(CH₂)ₙCN, —(CH₂)ₙO(CH₂)ₘOR^{a}, —(CH₂)ₙCO₂R^{a}, —(CH₂)ₙC(O)R^{a}, —SO₂NRₓR_{y}, —SO₂(CH₂)ₚCH₃, —CH═CHC(O)NRₓR_{y}, —CH═CHCN, —CH═CHCO₂R^{a}, —CO₂R^{a}, —C(O)R^{a}, —C(O)NRₓR_{y} and C₂₋₅alkenyl;
n and m are independently 1-4; and
p is 0-4.

Referring to the general formula (I), alkyl, alkylene and alkoxy include both straight and branched chain saturated hydrocarbon groups. Examples of alkyl groups include methyl and ethyl groups, examples of alkylene groups include methylene and ethylene groups, whilst examples of alkoxy groups include methoxy and ethoxy groups.

Referring to the general formula (I), alkenyl includes both straight and branched chain saturated hydrocarbon groups containing one double bond. Examples of alkenyl groups include ethenyl or n-propenyl groups.

Referring to the general formula (I), acyl refers to aliphatic or cyclic hydrocarbons attached to a carbonyl group through which the substituent bonds, such as acetyl.

Referring to the general formula (I), phenyl fused by a C₃₋₈cycloalkyl includes bicyclic rings such as 1,2,3,4-tetrahydronaphthyl, which, for the avoidance of doubt, is linked to the rest of the molecule through the aromatic ring.

Referring to general formula (I), a halogen atom includes fluorine, chlorine, bromine or iodine.

Referring to the general formula (I), C₁₋₃perfluoroalkyl and C₁₋₃perfluoroalkoxy includes compounds in which the hydrogens have been partially or fully replaced by fluorines, such as trifluoromethyl and trifluoromethoxy or trifluoroethyl.

Referring to the general formula (I), a 5-6 membered heteroaromatic group includes a single aromatic ring system containing at least one ring heteroatom independently selected from O, N and S. Suitable examples include pyridyl and thiazolyl.

Referring to the general formula (I), a 3-7 membered heterocyclyl group means any single ring system containing at least one ring heteroatom independently selected from O, N and S, wherein said ring is saturated, unsaturated or aromatic.

Preferably Ar₁ is phenyl, naphthyl, 1,2,3,4-tetrahydronaphthyl, indolyl, benzofuranyl, benzothiophenyl or indazolyl. More preferably Ar₁ is phenyl, 1,2,3,4-tetrahydronaphthyl or indolyl. More preferably still, Ar₁ is phenyl, 1,2,3,4-tetrahydronaphthyl or indolyl.

Where Ar₁ is 1,2,3,4-tetrahydronaphthyl, the link to the piperidine ring is preferably through the 2-position of the 1,2,3,4-tetrahydronaphthyl moiety and mono-substitution by R¹ is in the corresponding 1-position.

Where Ar₁ is indolyl, the link to the piperidine ring is preferably through the 3-position of the indole moiety and mono-substitution by R¹ is in the corresponding 1-position.

Preferably E is n-butylene.

Preferably X is —NR^{a}CO—. Preferably R^{a} is hydrogen.

Preferably Ar₂ is phenyl or a 5-6-membered heteroaromatic group (more preferably phenyl, pyridyl, thiazolyl, oxazolyl, pyrazolyl or imidazolyl).

Preferably Ar₃ is phenyl, pyridyl, pyridazinyl, pyrimidinyl, furyl or thienyl, more preferably phenyl. Preferably Ar₃ is substituted by C₁₋₄alkylsulfonylamino (such as —NHSO₂CH₃, —NHSO₂CH(CH₃)₂), fluoroC₁₋₄alkylsulfonylamino (such as —NHSC₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbony), aminocarbonyl, C₁₋₄alkylaminocarbonyl or di-C₁₋₄alkylaminocarbonyl.

When Ar₃ is phenyl, para-substitution is preferred.

Preferably Ar₂ is optionally substituted by C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} (where Rₓ and R_{y} are independently hydrogen or C₁₋₄alkyl and n is 1-3) or —CO₂(CH₂)ₚCH₃ (where p is 0-3).

Particularly preferred compounds of the invention include those in which each variable in Formula (I) is selected from the preferred groups for each variable. Even more preferable compounds of the invention include those where each variable in Formula (I) is selected from the more preferred or most preferred groups for each variable.

Preferably
Ar₁ is phenyl, naphthyl, 1,2,3,4-tetrahydronaphthyl, indolyl, benzofuranyl, benzothiophenyl or indazolyl; where Ar₁ is optionally substituted by 1-4 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NR^{a}CO—;
R¹ is halogen, C₁₋₄alkoxy or C₁₋₄alkyl;
R^{a} is C₁₋₄alkyl or hydrogen;
Rₓ and R_{y} are independently hydrogen or C₁₋₄alkyl;
n is 1-3; and
p is 0-3.

More preferably
Ar₁ is phenyl, 1,2,3,4-tetrahydronaphthyl or indolyl; where Ar, is optionally substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl (preferably methoxy or methyl);
Rₓ and R_{y} are independently hydrogen or C₁₋₄alkyl;
n is 1-3; and
p is 0-3.

Particularly preferred groups of compounds of formula (I) are where:
(A) Ar₁ is phenyl, 1,2,3,4-tetrahydronaphthyl or indolyl; where Ar₁ is substituted by 1-2 R¹ groups which may be the same or different;
Ar₂ is phenyl, pyridyl, thiazolyl, oxazolyl, pyrazolyl or imidazolyl; each of which is substituted by 1-4 groups independently selected from the list: hydroxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl (preferably methoxy or methyl);
Rₓ and R_{y} are independently hydrogen or C₁₋₄alkyl;
n is 1-3; and
p is 0-3.
(B) Ar₁ is phenyl, 1,2,3,4-tetrahydronaphthyl or indolyl; where Ar₁ is optionally substituted by 1-2 R¹ groups which may be the same or different;
Ar₂ is pyridyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl (preferably methoxy or methyl);
Rₓ and R_{y} are independently hydrogen or C₁₋₄alkyl;
n is 1-3; and
p is 0-3.
(C) Ar₁ is phenyl, 1,2,3,4-tetrahydronaphthyl or indolyl; where Ar₁ is optionally substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl (preferably methoxy or methyl);
Rₓ and R_{y} are independently hydrogen or C₁₋₄alkyl;
n is 1-3; and
p is 0-3.
(D) Ar₁ is phenyl, 1,2,3,4-tetrahydronaphthyl or indolyl; where Ar₁ is optionally substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;

Ar₃ is pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl (preferably methoxy or methyl);
Rₓ and R_{y} are independently hydrogen or C₁₋₄alkyl;
n is 1-3; and
p is 0-3.

Preferred compounds according to the first aspect are selected from the list:
2-Hydroxymethyl-4′-trifluoromethyl-biphenyl4-carboxylic acid {4-[4-(1H-indol-3-yl)-piperidin-1 -yl]-butyl}-amide (Example 1);
2-(4-Cyano-phenyl)4-hydroxymethyl-thiazole-5-carboxylic acid {4-[4-(1-methoxy-5,6,7,8-tetrahydro-naphthalen-2-yl)-piperidin-1-yl]-butyl}-amide (Example 7);
2-(4-Chloro-phenyl)-4-hydroxymethyl-thiazole-5-carboxylic acid {4-[4-(1-methoxy-5,6,7,8-tetrahydro-naphthalen-2-yl)-piperidin-1-yl]-butyl)amide (Example 10);
5-(4-Cyano-phenyl)-2-(2-hydroxy-ethyl)-2H-pyrazole-3-carboxylic acid {4-[4-(1-methoxy-5,6,7,8-tetrahydro-naphthalen-2-yl)-piperidin-1-yl]-butyl}-amide (Example 21);
4-(5-Chloro-thiophen-2-yl)-N-{4-[4-( 1-methoxy-5,6,7,8-tetrahydro-naphthalen-2-yl) piperidin-1-yl-butyl}-benzamide (Example 23);
4-(5-Chloro-pyridin-2-yl)-N-{4-[4-(1-methoxy-5,6,7,8-tetrahydro-naphthalen-2-yl)-piperidin-1-yl]-butyl}-benzamide (Example 32);
4-(6-Chloro-pyridin-3-yl)-N-{4-[4-(1-methoxy-5,6,7,8-tetrahydro-naphthalen-2-yl)-piperidin-1-yl]-butyl}-benzamide (Example 34);
6-(4-Chloro-phenyl)-N-{4-[4-(1-methoxy-5,6,7,8-tetrahydro-naphthalen-2-yl)-piperidin-1-yl]-butyl)-nicotinamide (Example 38);
6-(4-Cyano-phenyl)-N-{4-[4-(1-methoxy-5,6,7,8-tetrahydro-naphthalen-2-yl)-piperidin-1-yl]-butyl}-nicotinamide (Example 39);
6-(5-Chloro-thiophen-2-yl)-N-{4-[4-(1-methoxy-5,6,7,8-tetrahydro-naphthalen-2-yl)-piperidin-1-yl]-butyl}-nicotinamide (Example 40); and
2-(4-chlorophenyl)-1,4-dimethyl-1H-imidazole-5-carboxylic acid {4-[4-(1-methoxy-5,6,7,8-tetrahydro-naphthalen-2-yl)-piperidin-1-yl]-butyl}-amide (Example 45).

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

As used herein the term “physiologically acceptable” means a compound which is suitable for pharmaceutical use.

Suitable physiologically acceptable salts of the compounds of general formula (I) include acid addition salts formed with pharmaceutically acceptable inorganic acids for example, phosphates, hydrochlorides, hydrobromides or sulphates, or with pharmaceutically acceptable organic acids for example mesylates, lactates and acetates. More suitably, a physiologically acceptable salt of the compounds of general formula (I) is a phosphate salt.

The solvates may, for example, be hydrates.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like.

Hereinafter, compounds, their pharmaceutically acceptable salts, their solvates and polymorphs, defined in any aspect of the invention (except intermediate compounds in chemical processes) are referred to as “compounds of the invention”.

Compounds of the invention may be administered as the raw chemical but the active ingredient is preferably presented as a pharmaceutical formulation.

Compounds of the invention may be formulated for oral, buccal, parenteral, transdermal, topical (including ophthalmic and nasal), depot or rectal administration or in a form suitable for administration by inhalation or insufflation (either through the mouth or nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled release of the active compound.

For buccal administration the composition may take the form of tablets or lozenges formulated in conventional manner.

For transdermal administration the compounds of the invention may be formulated as creams, gels, ointments or lotions or as a transdermal patch. Such compositions may for example be formulated with an aqueous or oily base with the addition of suitable thickening, gelling, emulsifying, stabilising, dispersing, suspending, and/or colouring agents.

The compounds of the invention may be formulated for parenteral administration by bolus injection or continuous infusion. Formulations for injection may be presented in unit dosage form e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds of the invention may be formulated for topical administration in the form of ointments, creams, gels, lotions, pessaries, aerosols or drops (e.g. eye, ear or nose drops). Ointments and creams may, for example, be formulated with an aqueous or oily base with the addition of suitable thickening and/or gelling agents. Ointments for administration to the eye may be manufactured in a sterile manner using sterilised components.

Lotions may be formulated with an aqueous or oily base and will in general also contain one or more emulsifying agents, stabilising agents, dispersing agents, suspending agents, thickening agents, or colouring agents. Drops may be formulated with an aqueous or non aqueous base also comprising one or more dispersing agents, stabilising agents, solubilising agents or suspending agents. They may also contain a preservative.

The compounds of the invention may also be formulated in rectal compositions such as suppositories or retention enemas, e.g. containing conventional suppository bases such as cocoa butter or other glycerides.

The compounds of the invention may also be formulated as depot preparations. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

For intranasal administration, the compounds of the invention may be formulated as solutions for administration via a suitable metered or unit dose device or alternatively as a powder mix with a suitable carrier for administration using a suitable delivery device.

The compositions may contain from 0.1% upwards, e.g. 0.1-99% of the active material, depending on the method of administration. A proposed dose of the compounds of the invention is 0.25 mg/kg to about 125 mg/kg bodyweight per day e.g. 20 mg/kg to 100 mg/kg per day. It will be appreciated that it may be necessary to make routine variations to the dosage, depending on the age and condition of the patient and the precise dosage will be ultimately at the discretion of the attendant physician or veterinarian. The dosage will also depend on the route of administration and the particular compound selected.

The compounds of the invention may, if desired, be administered with one or more therapeutic agents and formulated for administration by any convenient route in a conventional manner. Appropriate doses will be readily appreciated by those skilled in the art. For example, the compounds of the invention may be administered in combination with an HMG CoA reductase inhibitor, an agent for inhibition of bile acid transport or fibrates.

The compounds of the invention are inducers of LDL-r expression and are thus of use in the treatment of conditions resulting from elevated circulating levels of LDL-cholesterol. Thus compounds of the invention are of use in the treatment of diseases in which lipid imbalance is important, e.g. atherosclerosis, pancreatitis, non-insulin dependent diabetes mellitus (NIDDM), coronary heart diseases and obesity. In addition compounds of the invention are also useful in lowering serum lipid levels, cholesterol and/or triglycerides, and are of use in the treatment of hyperlipemia, hyperlipidemia, hyperlipoproteinemia, hypercholesterolemia and/or hypertriglyceridemia.

It will be appreciated that reference to treatment is intended to include prophylaxis as well as the alleviation of established symptoms.

Compounds of the invention may be prepared in a variety of ways. In the following reaction schemes and hereafter, unless otherwise stated groups Ar₁, Ar₂, Ar₃, R¹, R^{a}, E and X are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc . . . (IVa), (IVb), (IVc) etc.

Compounds of formula (Ia), i.e. compounds of formula (I) where X is —NR^{a}C(O)— where the nitrogen is attached to E, may be prepared according to reaction scheme 1 by reacting compounds of formula (II) with compounds of formula (III) where L is a leaving group such as halogen or hydroxy, using standard amide coupling conditions detailed in the experimental section.

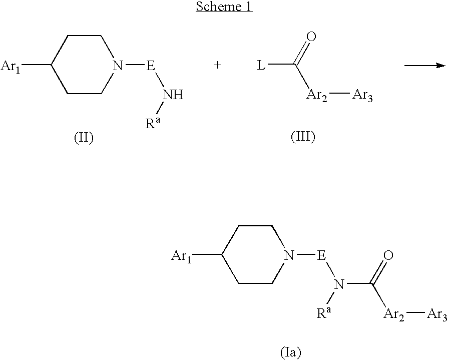

Compounds of formula (Ib), i.e. compounds of formula (I) where R¹ is —OR, may be prepared from the corresponding hydroxy compound (IV) according to reaction scheme 2. Preferred reaction conditions comprise treating (IV) with a suitable base such as sodium hydride or caesium carbonate followed by addition of RL where L is a leaving group such as halogen. Compounds of formula (IV) may be prepared by adapting methods described herein for the preparation of compounds of formula (I).

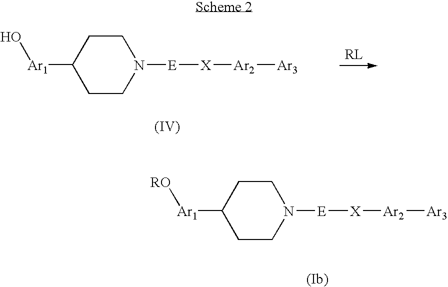

Compounds of formula (I) where Ar₁ is a nitrogen containing heterocycle may be substituted on the nitrogen by nucleophilic substitution. For instance where Ar₁ is indol-3-yl, substitution may be effected by treating (I) with base such as sodium hydride followed by reaction with a suitable nucleophile.

Compounds of formula (I) may be prepared be coupling boronic acid compounds of formula (V) with compounds of formula (VI) according to reaction scheme 3. Preferred reaction conditions comprise treatment with Pd(PPh₃)₄ and a suitable base such as sodium carbonate at elevated temperature.

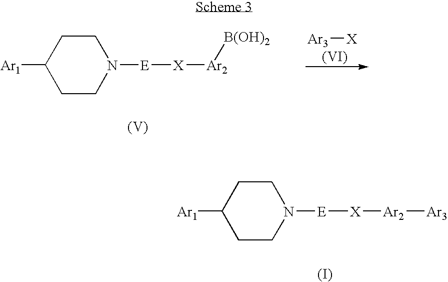

Compounds of formula (II) may be prepared according to reaction scheme 4 by reacting a compound of formula (VII) with a compound of formula (Vil) where L is a leaving group such as halogen and P is a suitable protecting group. Preferred conditions comprise reaction with a suitable base such as potassium carbonate. Removal of protecting group P gives compounds of formula (II). A preferred nitrogen protecting group is where the nitrogen attached to E and group R² form phthalimide (i.e. 1,3-dioxo-1,3-dihydro-isoindol-2-yl). Removal of the phthalimide protecting group gives compounds of formula (II) where R² is hydrogen. Preferred conditions comprise treatment with hydrazine at elevated temperature.

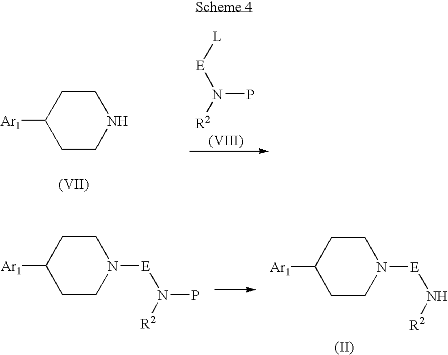

Compounds of formula (VII) may be prepared by methods described in the experimental section hereinbelow. Compounds of formula (VIII) are either known or may be prepared from known compounds by methods available to the skilled person.

Compounds of formula (IIIa), i.e. compounds of formula (III) (see reaction scheme 1) where L is hydroxy, may be prepared according to reaction scheme 5 by coupling boronic acid compounds of formula (IX) with compounds of formula (X) where L is a leaving group such as halogen under analogous conditions described for reaction scheme 3.

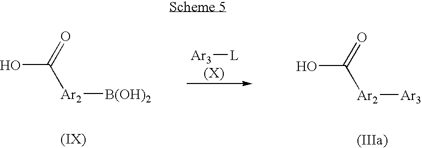

Compounds of formula (IX) and (X) are either known or may be prepared from known compounds by methods available to the skilled person.

The protecting groups used in the preparation of compounds of formula (I) may be used in conventional manner. See for example ‘Protective Groups in Organic Chemistry’ Ed. J. F. W. McOmie (Plenum Press 1973) or ‘Protective Groups in Organic Synthesis’ by Theodora W Greene and P M G Wuts (John Wiley and Sons 1991). Conventional amino protecting groups may include for example aralkyl groups, such as benzyl, diphenylmethyl or triphenylmethyl groups; and acyl groups such as N-benzyloxycarbonyl or t-butoxycarbonyl. Conventional carboxylic acid protecting groups include methyl and ethyl groups.

It will be appreciated that the invention includes the following further aspects. The preferred embodiments described for the first aspect extend these further aspects:

i) a pharmaceutical composition comprising a compound of the invention and a pharmaceutically acceptable carrier or diluent;

ii) the use of a compound of the invention in the manufacture of a medicament for use in the treatment of conditions resulting from elevated circulating levels of LDL-cholesterol;

iii) the use of a compound of the invention in the manufacture of a medicament for the treatment or prophylaxis of a disorder in which lipid imbalance is important(such as atherosclerosis, pancreatitis, non-insulin dependent diabetes mellitus (NIDDM), coronary heart diseases and obesity);

iv) the use of a compound of the invention in the manufacture of a medicament for lowering serum lipid levels, cholesterol and/or triglycerides;

v) the use of a compound of the invention in the manufacture of a medicament for the treatment or prophylaxis of hyperlipemia, hyperlipidemia, hyperlipoproteinemia, hypercholesterolemia and/or hypertriglyceridemia;

vi) a compound of the invention for use as a medicament;

vii) a method of treatment or prophylaxis of a disorder resulting from elevated circulating levels of LDL-cholesterol in a human patient comprising administering to the human an effective amount of a compound of the invention;

viii) a method of lowering serum lipid levels, cholesterol and/or triglycerides in a human patient comprising administering to the human an effective amount of a compound of the invention; and

ix) a combination of a compound of the invention with an HMG CoA reductase inhibitor, an agent for inhibition of bile acid transport or a fibrate.

According to a further aspect, the invention provides a compound of formula (I) 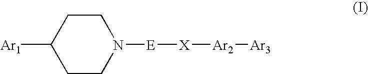
wherein
Ar₁ represents
   (i) phenyl, naphthyl, or phenyl fused by a C₃₋₈cycloalkyl , or
   (ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, provided that at least one ring is aromatic,
where Art optionally bears 1-4 groups independently represented by R¹;
R¹ is selected from halogen, —O—(C₀₋₄ alkylene)-R² or —(C₀₋₄alkylene)-R², where each alkylene group may additionally incorporate an oxygen in the chain, with the proviso that there are at least two carbon atoms between any chain heteroatoms;
R² represents
   (i) hydrogen, C₁₋₄ perfluoroalkyl, C₁₋₄perfluoroalkoxy.
   (ii) phenyl, phenyl fused by a C₃₋₈cycloalkyl , naphthyl or a 5- or 6-membered heteroaromatic group, optionally substituted by one or two groups independently selected from halogen, C₁₋₄ alkyl, hydroxy, C₁₋₄ alkoxy, amino, C₁₋₄ alkylamino and di-C₁₋₄ alkylamino,
   (iii) C₃₋₈cycloalkyl or a monocyclic heterocyclyl radical containing a total of 3-7 ring atoms, wherein said radical contains a total of from 1-4 ring heteroatoms independently selected from oxygen, nitrogen and sulfur, wherein said radical may be independently saturated, partially unsaturated, or aromatic, and where the C₃₋₈cycloalkyl or a monocyclic heterocyclyl may bear one or two groups independently selected from halogen, C₁₋₄ alkyl, hydroxy, C₁₋₄ alkoxy, amino, C₁₋₄ alkylamino and di-C₁₋₄ alkylamino, or
   (iv) amino, C₁₋₄ alkylamino or di-C₁₋₄alkylamino;
Ar₂ represents phenyl or a 5-6 membered heteroaromatic group or a bicyclic heteroaromatic group, where each group is substituted by 1-4 groups independently selected from the group consisting of: (CH₂)ₙOH and C(O)O(CH₂)ₘCH₃, wherein n is 1-4 and m is 0-4;
Ar₃ represents
   (i) phenyl, naphthyl, or phenyl fused by a C₃₋₈cycloalkyl,
   (ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
where Ar₃ is optionally substituted by 1-4 groups independently selected from the group consisting of: hydroxy, alkyl, C₁₋₄ alkoxy, C₂₋₄ alkenyl, C₂₋₄ alkenyloxy, C₁₋₄perfluoroalkoxy, C₁₋₄ acylamino or an electron withdrawing group selected from the list consisting of: nitrile, nitro, C_{1-4,} C₁₋₄ perfluoroalkyl, C₁₋₄acyl , C₁₋₄ alkoxycarbonyl, aminocarbonyl, C₁₋₄ alkylaminocarbonyl; di-C₁₋₄alkylaminocarbonyl, C₁₋₄ alkylsulfonyl, C₁₋₄ alkylaminosulfonyl and di-C₁₋₄alkylaminosulfonyl, C₁₋₄ alkylsulfonyl and C₁₋₄ alkylsulfoxy;
E represents —C₁₋₆ alkylene-;
X represents —CON(H or C₁₋₄alkyl)- or —N(H or C₁₋₄alkyl)CO—;
or a physiologically acceptable prodrug, salt or solvate thereof.

The invention is further described with reference to the following non-limiting examples.

Abbreviations

Pd(PPh₃)₄—Tetrakis-(triphenylphosphine)-palladium(0), THF—Tetrahydrofuran, BF₃—Et₂O—Boron trifluoride diethyl etherate, DCM—Dichloromethane, TEA—triethylamine, CH₃CN—Acetonitrile, EtOH—Ethanol, EtOAc—Ethyl acetate, iPr₂O—Di-isopropyl ether, iPrOH—Isopropanol, Pd/C—Palladium on carbon, Et₂O—diethyl ether, Chex—cyclohexane, MeOH—Methanol, DMF—Dimethyl formamide, EDCl—1-(3-dimethylaminopropyl)-, ethylcarbodiimide hydrochloride, HOBt—1-Hydroxybenzotriazole, rt—Room temperature, AcOH—Acetic acid, NaOH—Sodium hydroxide, KOH—potassium hydroxide, HCl—Hydrochloric acid, HBr—Bromhydric acid, AcOH—Acetic acid, NaH—Sodium hydride, Na₂SO₄—Sodium sulfate, CCl₄—Carbon tetrachloride, AlBN—2,2′-Azobis(2-methylpropionitrile), K₂CO₃—Potassium carbonate, Na₂CO₃—Sodium carbonate, NaCl—Sodium chloride, POCl₃—Phosphorus oxychloride, DME—Ethylene glycol dimethyl ether, Cs₂CO₃—Cesium carbonate, CrO₃—Chromium(VI) oxide, BBr₃—Boron tribromide, LiOH, H₂O—Lithium hydroxyde, monohydrate, Mg—Magnesium, NaOAc—Sodium Acetate, NBS—N Bromo succinimide, NaHCO₃—Sodium hydrogeno carbonate, Nal—Sodium iodide, TMOF—Trimethyl ortho formate, NaHB(OAc)₃—Sodium triacetoxy borohydride, CuCN—Copper cyanide, FeCl₃—Iron chloride, LiHMDS—Lithium hexamethyidisilazide, H₂SO₄—sulfuric acid.

### Intermediate 1: 2-Methyl-4′-trifluoromethyl-biphenyl-4-carboxylic acid methyl ester

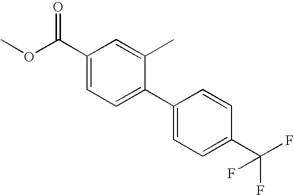

To a solution of 4-bromo-3-methyl-benzoic acid methyl ester (10.1 g , 44 mmol) in acetonitrile (300 mL) was added Pd(PPh₃)₄ (1 g), a 2M Na₂CO₃ solution (40 mL) and 4-trifluoromethyl boronic acid (9.21 g, 1.1 eq). The mixture was stirred to reflux for 24 hours. After cooling, the reaction was evaporated, diluted with water and extracted with DCM. The organic layer was dried over Na₂SO₄, filtered through silica and evaporated. The title compound (11.9 g, 40.5 mmol) was obtained as a brown solid in a 94.5% yield; GC/MS: M⁺ C₁₆H₁₃F₃O₂ 294

### Intermediate 2: 2-Bromomethyl-4′-trifluoromethyl-biphenyl-4-carboxylic acid methyl ester

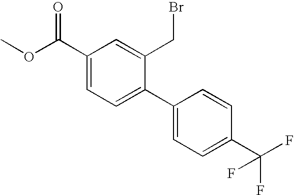

To a solution of intermediate 1 (11.9 g, 40.5 mmol) in CCl₄ (300 mL) was added N-bromosuccinimide (10.8 g, 1.5 eq.) and AlBN (400 mg). The mixture was stirred at reflux for 48 hours. After cooling water was added and the organic layer was separated and dry over Na₂SO₄ to give after evaporation, the title compound as a yellow oil. The compound was used in the next step without further purification; GC/MS: M⁺ C₁₆H₁₂BrF₃O₂ 373

### Intermediate 3: 2-Acetoxymethyl-4′-trifluoromethyl-biphenyl4-carboxylic acid methyl ester

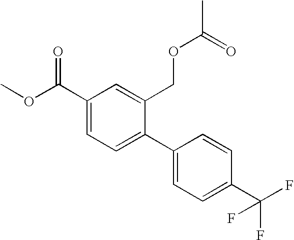

To a solution of intermediate 2 (16.0 g, 43 mmol) in DMF (400 mL) and acetonitrile (2 mL) was added sodium iodide (small quantity) and sodium acetate (10.6 g, 3 eq.). The reaction was stirred to reflux for 24 hours and then water (10 mL) was added and the mixture evaporated. The residue was diluted with water and extracted with DCM. The organic layer was dried over Na₂SO₄ and evaporated. After purification by flash chromatography using EtOAc/Cyclohexane (10/90) as eluent, the title compound (6.0 g, 17 mmol) was obtained as white crystals in a 42% yield; m.p. 94° C.

### Intermediate 4: 2-Hydroxymethyl-4′-trifluoromethyl-biphenyl4-carboxylic acid

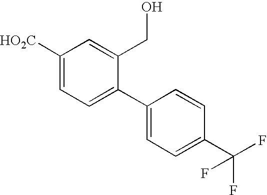

To a solution of intermediate 3 (6.0 g, 17 mmol) in MeOH (200 mL) was added a 1 N NaOH solution (51 mL, 3 eq.). The mixture was stirred at reflux for 24 hours and the solvent was evaporated. The residue was treated with a 1N HCl solution (60 mL) and the precipitate was filtered, washed with water and dried over Na₂SO₄ to give the title compound (5.0 g, 16.9 mmol) as a white powder in a quantitative yield; LC/MS: M−H C₁₅H₁₀F₃O₃ 295.

### Intermediate 5:1 [4-( 1-Methyl-1H-indol-3-yl)-Piperidin-1-yl]-ethanone

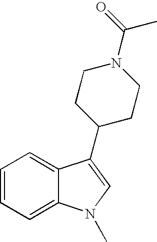

To a solution of 1-[4-(1H-indol-3-yl)-piperidin-1-yl]-ethanone, (4.95 g, 20.0 mmol) in dry DMF (100 mL) was added NaH 60% (0.98 g, 1.2 eq.) and methyl iodide (1.52 mL, 1.2 eq.). The mixture was stirred at rt for 18 hours. The mixture was evaporated, diluted with water, extracted (DCM) and dried over Na₂SO₄ to give after evaporation, the title compound (5.2 g, 20.0 mmol) as an oil in quantitative yield; LC/MS: M+H C₁₆H₂₁N₂O 257.

### Intermediate 6: 1-Methyl-3-piperidin4-yl-1H-indole

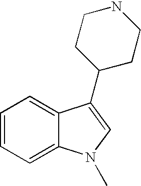

To a solution of intermediate 5 (5.2 g, 20.0 mmol) in EtOH (100 mL) was added a NaOH/H₂O (1/1) solution (14 mL) and the reaction was stirred at reflux for 16 hours. After cooling, the reaction was concentrated in vacuo, and the residue diluted with water and extracted with DCM. The organic phase was then dried over Na₂SO₄ and evaporated to give the title compound as a yellow solid in a quantitative yield; LC/MS: M+ C₁₄H₁₈N₂ 214.

### Intermediate 7: 2-{4-[4-(1-Methyl-1H-indol-3-yl)-piperidin-1-yl]-butyl}-isoindole-1,3-dione

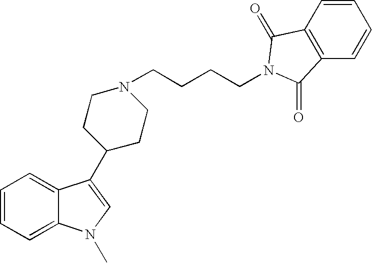

To a solution of intermediate 6 (5.0 g, 23 mmol) in acetone (200 mL) was added potassium carbonate (6.35 g, 2.0 eq.) and N4-bromobutyl phthalimide (7.25 g, 1.1 eq.) and the reaction was stirred at reflux for 16 hours. After cooling, the reaction was filtered and the solvent was removed in vacuo. After purification by flash chromatography using DCM/MeOH (95/5) as eluent, the title compound was obtained as a yellow oil a quantitative yield; LC/MS: M+H C₂₆H₂₇N₃O₂ 416.

### Intermediate 8: 4-[4-(1-Methyl-1H-indol-3-yl)-piperidin-1-yl]-butylamine

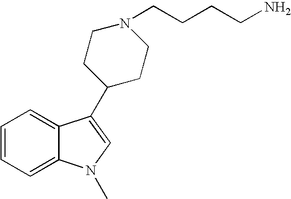

To a solution of intermediate 7 (9.6 g, 23 mmol) in MeOH (200 mL) was added hydrazine hydrate (4.6 mL, 4.0 eq) and the reaction stirred at reflux for 16 hours. After evaporation under reduced pressure, the residue was taken up in water and treated with a concentrated HCl solution until pH=3-4. The white precipitate was filtered and washed with water. The filtrate was treated with a concentrated NaOH solution until pH>12. Extraction with DCM, drying over Na₂SO₄ and filtration gave the title compound (5.5 g, 19 mmol) as a yellow oil in a 83% yield; GC/MS: M⁺ C₁₈H₂₇N₂ 285.

### Intermediate 9: 4-Trifluoromethyl-thiobenzamide

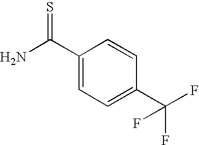

A solution of α,α,α-trifluoro-p-tolunitrile (603.5 g, 3.53 mol) in dry DMF (2 L) under N₂ was heated at 70° C. and thioacetamide (505 g, 1.9 eq.) added. The reaction mixture was treated with HCl gas for 15 minutes and was stirred at 95° C. for 6 hours. This treatment was repeated 3 times and the mixture stirred at rt for 24 hours. After cooling to 0° C., water was added and the residue was extracted with diethyl ether (4 L). The organic layer was washed with water (3 L), dried over Na₂SO₄ and evaporated. The brownish powder was washed with pentane (3 L) to give the title compound (530.3 g, 2.59 mol) as a brown solid in 73% yield; GC/MS: M⁺ C₈H₆F₃NS 205.

### Intermediate 10: 4-Methyl-2-(4-trifluoromethyl-phenyl)-thiazole-5-carboxylic acid ethyl ester

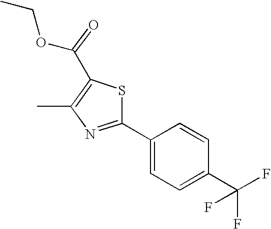

To a solution of intermediate 9 (530.3 g, 2.59 mol) in EtOH (2.6 L) was added 2-chloro-3-oxo-butyric acid ethyl ester (465 mL. 1.3 eq) and the mixture was stirred at rt for 7 hours and at 70° C. for 14 hours. After cooling to 0° C., the precipitate was filtered and washed with cold EtOH (500 mL) to give the title compound (573.0 g, 1.89 mol) as a beige powder in a 73% yield; ¹H NMR (CDCl₃, 300 MHz) [symbol] 7.9 (d, 2H), 7.6 (d, 2H), 4.3 (q, 2H), 2.65 (s, 3H), 1.25 (t. 3H).

### Intermediate 11: 4-Bromomethyl-2-(4-trifluoromethyl-phenyl)-thiazole-5-carboxylic acid ethyl ester

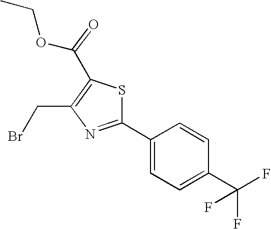

To a solution of intermediate 10 (15.75 g, 50.0 mmol) in CCl₄ was added slowly t N-bromosuccinimide (8.9 g, 1.1 eq.) and AlBN (1 g, 10% mol) and the mixture was stirred at 80° C. for 3 hours. On cooling the mixture was filtered and the filtrate evaporated. After purification by flash chromatography, using DCM/cyclohexane (60/40) as eluent, the title compound (4.9 g, 12.5 mmol) was obtained as a white solid in 25% yield; ¹H NMR (CDCl₃, 300 MHz) [symbol] 8.2 (d, 2H), 7.8 (d, 2H), 5.1 (s, 2H), 4.5 (q, 2H), 1.3 (t, 3H).

### Intermediate 12: 4-Acetoxymethyl-2-(4-trifluoromethyl-phenyl)-thiazole-5-carboxylic acid ethyl ester

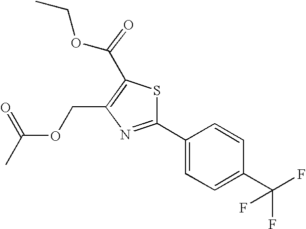

To a solution of intermediate 11 (4.9 g, 12.5 mmol) in AcOH (15 mL) was added sodium acetate (2.0 g, 2 eq.) and the mixture was stirred at reflux for 14 hours. After cooling at rt, the mixture was diluted with water (150 mL) and extracted with diethyl ether (250 mL). The organic layer was washed with a 1N NaOH solution, dried over Na₂SO₄ and evaporated. The title compound (3.24 g, 8.7 mmol) was obtained as white crystals in a 72% yield; m.p. 82° C.

### Intermediate 13: 4-Hydroxymethyl-2-(4-trifluoromethyl-phenyl)-thiazole-5-carboxylic acid

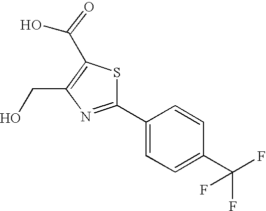

To a solution of intermediate 12 (3.24 g, 8.7 mmol) in EtOH/H₂O (40 mL/20 mL) was added NaOH (1.4 g, 4 eq.) and the mixture stirred at reflux for 2 hours. After partial evaporation, water (100 mL) was added and the mixture treated with a concentrated HCl solution to obtain pH=1. The mixture was filtered and the filter cake was washed with water and dried to give the title compound (2.38 g, 7.8 mmol) as a white solid in 90% yield; m.p. 250-252° C.

### Intermediate 14: 1-[4-(1-Hydroxy-5,6,7,8-tetrahydro-naphthalen-2-yl)-3,6-dihydro-2H-pyridin-1-yl]-ethanone

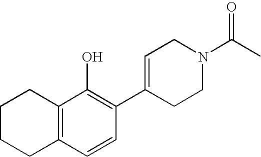

To a solution of 5,6,7,8-tetrahydro-naphthalen-1-ol (20.0 g, 0.135 mol) and 1-acetyl-4-piperidone (22.84 g, 1:2 eq.) in THF (400 mL), was added dropwise BF₃-Et₂O (68 mL, 4.0 eq). The mixture was stirred at 100° C. for 2 hours, and 14 hours at room temperature. The mixture was treated with a 1N HCl solution (400 mL). The resulting solution was extracted with DCM. The organic layer was dried over Na₂SO₄ and evaporated to dryness to give an oil which was recrystallized in acetonitrile to give the title compound (24.2 g, 89 mmol) as white crystals in 66% yield. GC/MS: M⁺ C₁₇H₂₀NO₂ 271

### Intermediate 15:1 -[4-(1-Hydroxy-5,6,7,8-tetrahydro-naphthalen-2-yl)-piperidin-1-yl-ethanone

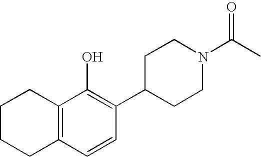

To a solution of intermediate 14 (9.4 g, 34.7 mmol) in EtOH (300 mL) was added Pd/C, 10% (0.9 g) and the reaction was stirred under an atmospheric pressure of hydrogen at 25° C. for 24 hours. The mixture was filtered through a bed of celite and the filtrate was evaporated under reduced pressure to give the title compound (9.6 g, 35 mmol) as a white foam; GC/MS: M⁺ C₁₇H₂₂NO₂ 273.

### Intermediate 16: 1-[4-(1-Methoxy-5,6,7,8-tetrahydro-naphthalen-2-yl)-piperidin-1-yl-ethanone

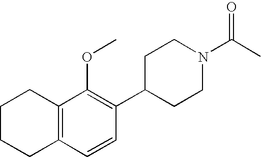

To a solution of intermediate 15 (9.6 g, 35 mmol) in DMF (300 mL) was added NaH 60% (1.6 g, 1.2 eq.) and iodomethane (22 mL,10 eq.) and the mixture was stirred at 60° C. for 2 hours. Water (10 mL) was added and the mixture was evaporated, the residue was taken in water and extracted with DCM and dried over Na₂SO₄ and give the title compound (10.2 g, 35 mmol) as a yellow oil in quantitative yield; GC/MS: M⁺ C₁₈H₂₄NO₂ 287.

### Intermediate 17: 4-(1-Methoxy-5,6,7,8-tetrahydro-naphthalen-2-yl)-piperidine

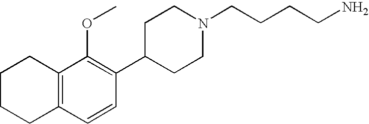

To a solution of intermediate 16 (10.2 g, 35 mmol) in EtOH (200 mL) was added a NaOH/H₂O (20 mL/20 mL) solution and the mixture was stirred to reflux for 24 hours. The solvent was evaporated, water was added and the residue was extracted with DCM. The organic layer was dried over Na₂SO₄ and evaporated to give the title compound (7.7 g, 31 mmol) as yellow oil in a 88.5% yield; GC/MS: M⁺ C₁₆H₂₂NO 245.

### Intermediate 18: 2-{4-[4-(1-Methoxy-5,6,7,8-tetrahydro-naphthalen-2-yl)-piperidin-1-yl]-butyl}-isoindole-1,3-dione

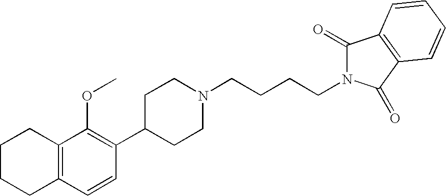

To a solution of intermediate 17 (7.7 g, 31 mmol) in acetone (200 mL) was added K₂CO₃ (8.55 g. 2 eq.) and 4-bromobutyl-phthalimide (8.86 g, 1 eq.) and the mixture was stirred at reflux for 6 hours. On cooling the mixture was filtered, the filtrate evaporated and the residue diluted in DCM. The misture was filtered through a bed of silica to give the title compound (12.2 g, 27 mmol) as a yellow oil in 87% yield: LC/MS: M+H C₂₈H₃₄N₂O₃ 447.

### Intermediate 19: 4-[4-(1-Methoxy-5,6,7,8-tetrahydro-naphthalen-2-yl)-piperidin-1-yl]-butylamine

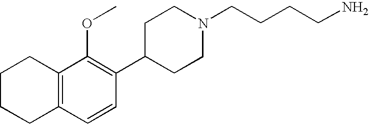

A solution of intermediate 18 (12.2 g, 27 mmol) in MeOH (200 mL) was treated with hydrazine monohydrate (5.5 mL, 4 eq.) and the resulting mixture was stirred at reflux for 16 hours. After cooling to rt, and evaporation under reduced pressure the residue was taken up in water and a 1N HCl solution was added until pH=4. Filtration gave a yellow solution which was treated with concentrated NaOH solution. Extraction with DCM, drying over Na₂SO₄ and filtration gave the title compound (6.97 g, 22 mmol) as a yellow oil in 81% yield; LC/MS: M+H C₂₀H₃₃N₂O 317.

### Intermediate 20: 1-[4-(4-Ethyl-2-hydroxy-phenyl)-3,6-dihydro-2H-pyridin-1-yl]-ethanone

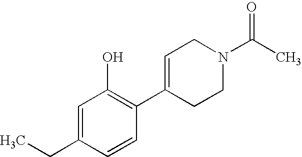

The same method was employed as in the preparation of intermediate 14 but starting from 3-ethyl-phenol and gave the title compound as a pink solid in quantitative yield; GC/MS: M⁺ C₁₅H₁₉NO₂ 245.

### Intermediate 21: 1-[4-(4-Ethyl-2-hydroxy-phenyl)-piperidin-1-yl]-ethanone

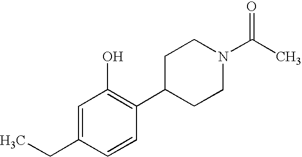

The same method was employed as in the preparation of intermediate 15 but starting from intermediate 20 and gave the title compound as a solid in a 89% yield; GC/MS: M⁺ C₁₅H₂₁NO₂ 247

### Intermediate 22: 1-[4-(2-Ethoxy-4-ethyl-phenyl)-piperidin-1-yl]-ethanone

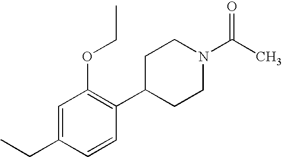

The same method was employed as in the preparation of Intermediate 16 but starting from intermediate 21 and ethyl iodide and gave the title compound as an oil in a quantitative yield; 1H NMR (CDCl₃, 300 MHz) [symbol] 6.9 (d, 1H), 6.6 (m, 2H), 4.7 (m, 1H), 4.0 (q, 2H), 3.8 (m, 1H), 3.1 (m, 2H), 2.5 (m, 3H), 2.05 (s, 3H), 1.7(m, 2H), 1.50 (m, 2H), 1.35 (t, 3H), 1.1 (t, 3H).

### Intermediate 23: 4-(2-Ethoxy-4-ethyl-phenyl)-piperidine

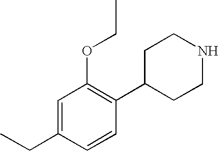

The same method was employed as in the preparation of intermediate 17 but starting from intermediate 22 and gave the title compound as a yellow oil in 94% yield; 1H NMR (CDCl₃, 300 MHz) [symbol] 67.1 (d, 1H), 6.7 (d, 1H), 4.7 (d, 1H), 4.05 (q, 2H), 3.1 (m, 2H), 3.05 (m, 1H), 2.7 (td, 2H), 2.55 (q, 2H), 1.75 (m, 3H), 1.55 (m, 2H), 1.35 (t, 3H), 1.1 (t, 3H).

### Intermediate 24: 2-{4-[4-(2-Ethoxy-4-ethyl-phenyl)-piperidin-1-yl]-butyl}-isoindole-1,3-dione

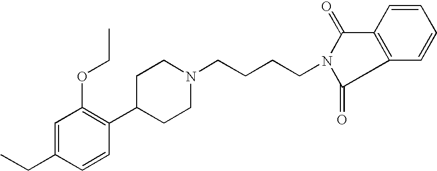

The same method was employed as in the preparation of intermediate 18 but starting from intermediate 23 and gave the title compound as a yellow oil in 97% yield; 1H NMR (CDCl₃, 300 MHz) [symbol] 7.8 (m, 2H), 7.6 (m, 2H), 7.0 (d, 1H), 6.65 (dd, 1H), 6.55 (sd, 1H), 3.95 (q, 2H), 3.65 (m, 3H), 2.95 (m, 2H), 2.8 (m, 1H), 2.5 (q, 2H), 2.4 (m, 2H), 2 ( td, 2H), 1.8-1.4 ( m, 8H), 1.3 (t, 3H), 1.15 (t, 3H).

### Intermediate 25: 4-[4-(2-Ethoxy-4-ethyl-phenyl)-piperidin-1-yl]-butylamine

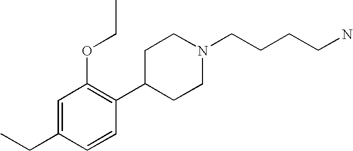

The same method was employed as in the preparation of intermediate 19 but starting from intermediate 24 and gave the title compound as a yellow oil in 81.5% yield; 1H NMR (CDCl₃, 300 MHz) [symbol] 7.1 (d, 1H), 6.7 (dd, 1H), 6.6 ( s, 1H), 4.0 (q, 2H), 3.0 (bd, 2H), 2.9 (m, 1H), 2.7 (t, 2H), 2.55 (q, 2H), 2.3 (m, 2H), 2.0 (td, 2H), 1.7-1.2 (m, 10H), 1.4 (t, 3H), 1.1 (t, 3H).

### Intermediate 26: 2-{4-[4-(1H-Indol-3-yl)-piperidin-1-yl1-butyl}-isoindole-1,3-dione

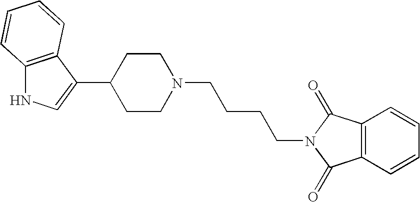

The same method was employed as in the preparation of intermediate 18 but starting from 3-piperidin-4-yl-1H-indole and gave the title compound as white crystals in a 77% yield after recrystallisation in CH₃CN; m.p. 106-108° C.

### Intermediate 27: 4-[4-(1H-Indol-3-yl)-piperidin-1-yl]-butylamine

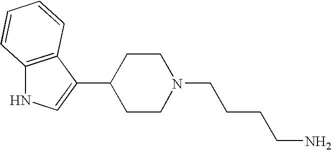

A solution of intermediate 26 (15 g, 37.5 mmol) in EtOH (250 mL) was treated with hydrazine monohydrate (2.5 mL, 1.4 eq.) and the resulting mixture was stirred at 55° C. for 16 hours. After evaporation under reduced pressure the residue was taken up in acetone. Filtration and evaporation of filtrate gave the title compound (10.5 g, 38.7 mmol) as an oil in a quantitative yield. The crude compound was used without further purification.

### Intermediate 28: 2-Bromo-4-methyl-thiazole-5-carboxylic acid ethyl ester

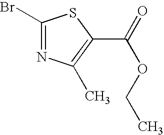

To a solution of 3-methyl-1-nitrosooxy-butane (19.8 mL, 2.1 eq) in acetonitrile (700 mL) was added at 0° C. trimethylsillyl bromide (19.6 mL, 2.1 eq) and the mixture was stirred at 0° C. for 20 min. A solution of 2-amino-4-methyl-thiazole-5-carboxylic acid ethyl ester (14.0 g, 1.0 eq) in acetonitrile/EtOAc:75/25 (700 mL) was added slowly at 0° C. After stirring overnight at rt, the reaction mixture was evaporated and purified by flash chromatography to give the title compound (13.2 g, 0.051 mol) as an orange solid in a 70% yield; GC/MS: M⁺ C₇H₈BrNO₂S 250; ¹H NMR (CDCl₃, 300 MHz) [symbol] 4.32 (q, 2H), 2.69 (s, 3H), 1.34 (t, 3H).

### Intermediate 29: 2-(4-Cyano-phenyl)-4-methyl-thiazole-5-carboxylic acid ethyl ester

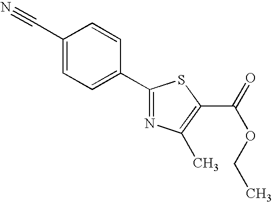

The same method was employed as in the preparation of intermediate 1 but starting from intermediate 28 (15.0 g, 0.06 mol) and 4-cyano-boronic acid (9.7 g, 1.1 eq) in DME, to give the title compound (5.2 g, 0.02 mol) as a solid in a 30% yield after purification by flash chromatography using DCM as eluent; m.p. 144° C.; ¹H NMR (CDCl₃, 300 MHz) [symbol] 8.07 (d, 2H), 7.73 (d, 2H), 4.36 (q, 2H), 2.78 (s, 3H), 1.38 (t, 3H).

### Intermediate 30: 4-Bromomethyl-2-(4-cyano-phenyl)-thiazole-5-carboxylic acid ethyl ester

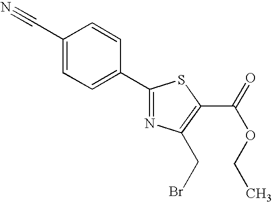

The same method was employed as in the preparation of intermediate 2 but starting from intermediate 29 (2.7 g, 0.01 mol) to give the title compound (1.72 g, 4.9 mmol) as a white solid in a 49% yield after purification by flash chromatography using DCM/Hexane (70/30); m.p. 156° C.

### Intermediate 31: 4-Acetoxymethyl-2-(4-cyano-phenyl)-thiazole-5-carboxylic acid ethyl ester

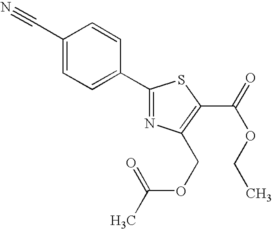

The same method was employed as in the preparation of intermediate 3 but starting from intermediate 30 (1.72 g, 5 mmol) to give the title compound (0.81 g, 2.5 mmol) as beige crystals in 50% yield after crystallisation in iPr₂O; m.p. 127° C.; ¹H NMR (CDCl_{3, 300} MHz) [symbol] 8.07 (d, 2H), 7.74 (d, 2H), 5.55 (s, 3H), 4.38 (q, 2H), 2.15 (s, 3H), 1.38 (t, 3H).

### Intermediate 32: 4-Hydroxymethyl-2-(4-cyano-phenyl)-thiazole-5-carboxylic acid ethyl ester

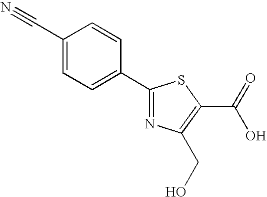

The same method was employed as in the preparation of intermediate 4 but starting from intermediate 31 (0.81 g, 2.5 mmol) to give the title compound (0.3 g, 1.15 mmol) as a beige solid in 46% yield after purification by flash chromatography using DCM/MeOH/AcOH (70/30/0.1%); m.p.>250° C.; LC/MS: M+H C₁₂H₉N₂O₃S 261.

### Intermediate 33: 5-Methyl-2-trifluoromethylsulfonyloxy-benzoic acid methyl ester

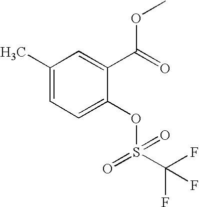

To an ice-cold solution of 2-hydroxy-5-methyl-benzoic acid methyl ester (15.0 g, 0.09 mol) in DCM and TEA (13.8 mL, 1.1 eq) was added slowly triflic anhydride (1.1 eq) and the reaction mixture was stirred at rt for 48 hours. Water was added, the product was extracted with DCM, the organic layer dried over Na₂SO₄, filtered and the solvent evaporated. Purification by flash chromatography on silica gel gave the title compound as a colorless oil (4.0 g, 13.4 mmol) in 15% yield; GC/MS: M⁺ C₁₀H₉F₃O₅S 298.

### Intermediate 34: 4-Methyl4′-trifluoromethyl-biphenyl-2-carboxylic acid methyl ester

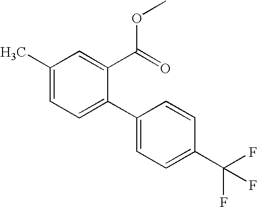

The same method was employed as in the preparation of intermediate 1 but starting from intermediate 33 and gave the title compound as a colorless oil in 33% yield; GC/MS: M⁺ C₁₆H₁₃F₃O₂ 294.

### Intermediate 35: 4′-Trifluoromethyl-biphenyl-2,4-dicarboxylic acid 2-methyl ester

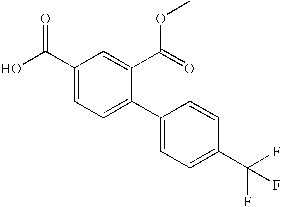

To a solution of intermediate 34 (1.3 g, 4.4 mmol) in acetic acid (20 mL) at 90° C., was added CrO₃ (2.2 g, 5 eq) and the reaction mixture was stirred at 90° C. for 21 hours. Water was added, the product extracted with DCM, dried over Na₂SO₄, filtered and the solvent evaporated. The title compound was obtained as green crystals (0.6 g, 1.85 mmol) in 42% yield; LC/MS: M−H C₁₆H₁₀F₃O₄ 323.

### Intermediate 36: 5-Methoxy-3-(1,2,3,6-tetrahydro-pyridin-4-yl)-1H-indole

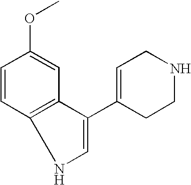

To a solution of 5-methoxy-1H-indole (50.0 g, 0.34 mol) in MeOH (500 mL) under N₂ was added 4-piperidone hydrate hydrochloride (104.5 g, 2.6 eq.) and KOH (56.0 g, 3 eq.) and the mixture was stirred at reflux overnight. Water (1 l) was added slowly over one hour and the precipitate filtered. The filter cake was washed with water, ethanol and diethyl ether to give the title compound (51.58 g, 0.23 mol) as a yellow solid in 58% yield; m.p. 184° C.

### Intermediate 37: 5-Methoxy-3-pyridin-4-yl-1H-indole

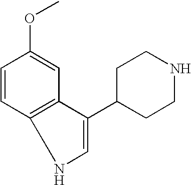

A solution of intermediate 36 (47.0 g, 0.21 mol) in EtOH (500 mL) was stirred with Pd/C 10% (4 g) under an atmospheric pressure of hydrogen for 24 hours. The reaction mixture was filtered through celite and the filtrate was evaporated to give the title compound (45.54 g, 0.2 mol) as an orange solid in 96% yield; ¹H NMR (CDCl₃, 300 MHz) [symbol] 7.4 (d, 1H), 7.3 (bd, 1H), 7.1 (s, 1H), 7.0 (ddp, 1H), 4.0 (s, 3H), 3.4 (dd, 2H), 2.9-3.0 (m, 3H), 2.2 (m, 2H), 1.9 (m, 2H).

### Intermediate 38: 2-{4-[4-(5-Methoxy-1H-indol-3-yl)-piperidin-1-yl]-butyl}-isoindole-1,3-dione

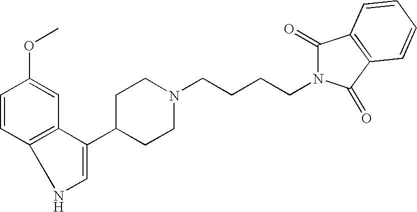

The same method was employed as in the preparation of intermediate 18 but starting from intermediate 37 and gave the title compound as a yellow powder in 70% yield; m.p. 155° C.

### Intermediate 39: 2-{4-[4-(1-Isopropyl-5-methoxy-1H-indol-3-yl)-piperidin-1-yl]-butyl}-isoindole-1,3-dione

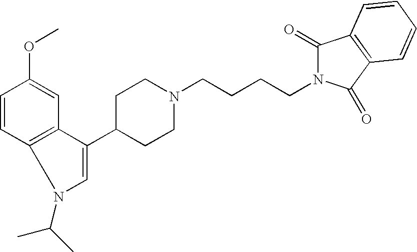

The same method was employed as in the preparation of intermediate 16 but starting from intermediate 38 and isopropyl bromide and gave the title compound as an oil in 23% yield; LC/MS: M+H C₂₉H₃₆N₃O₃ 474.

### Intermediate 40: 4-[4-(1-Isopropyl-5-methoxy-1H-indol-3-yl)-piperidin-1-yl]-butylamine

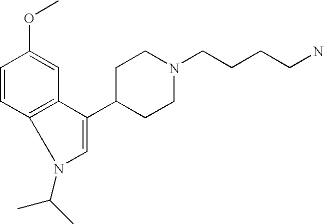

The same method was employed as in the preparation of intermediate 27 but starting from intermediate 39 and gave the title compound as a foam in quantitative yield, which was used in the next step without further purification.

### Intermediate 41: 4-Bromomethyl-2-(4-chloro-phenyl)-thiazole-5-carboxylic acid ethyl ester

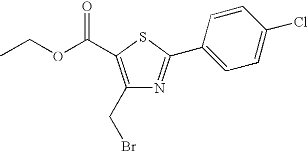

A solution of the available 2-(4-chloro-phenyl)-4-methyl-thiazole-5-carboxylic acid ethyl ester (3.0 g, 10.6 mmol) in CCl₄ (150 mL) was treated with NBS (2.84 g, 1.5 eq.) and AlBN and the mixture was heated under reflux for 6 hours. The cooled mixture was evaporated and water was added. The product was extracted with DCM, dried over Na₂SO₄, filtered and evaporated. The title compound was obtained as a yellow solid which was directly used in the next step without purification; GC/MS: M+ C₁₃H₁₁BrClNO₂S 360.

### Intermediate 42: 4-Acetoxymethyl-2-(4chloro-phenyl)-thiazole-5-carboxylic acid ethyl ester

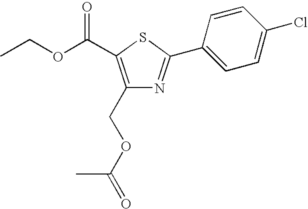

To a solution of intermediate 41 (4.45 g, 12.4 mmol) in CH₃CN (100 mL) and DMF (1 mL) was added NaOAc (3.1 g, 3 eq.) and Nal (small quantity) and the mixture was heated under reflux overnight. As the reaction was not completed, NaOAc (1.55 g, 1.5 eq.) was added and the mixture was heated under reflux for 24 hours. After evaporation, water was added and the product was extracted with DCM. The organic layer was dried over Na₂SO₄, filtered and evaporated. Purification by flash chromatography using DCM as eluent gave the title compound as a yellow solid (3.2 g, 9.44 mmol) in a 88% yield; GC/MS: M+ C₁₅H₁₄ClNO₄S 339.

### Intermediate 43: 2-(4-Chloro-phenyl)-4-hydroxymethyl-thiazole-5-carboxylic acid

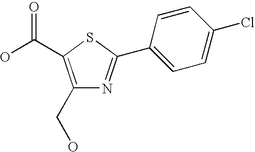

To a solution of intermediate 42 (3.2 g, 9.5 mmol) in EtOH (100 mL) was added a 1N NaOH solution (38 mL, 4 eq.) and the mixture was refluxed for 48 hours. After evaporation, the resulting mixture was treated with 1N HCl solution (200 mL). The resulting precipitate was filtered, washed with water and dried to give the title compound as a brown solid (2.6 g, 9.5 mmol) in a quantitative yield; LC/MS: M+H C₁₁H₉ClNO₃S 270.

### Intermediate 44: 2-(4-Cyano-phenyl)-4-methyl-thiazole-5-carboxylic acid

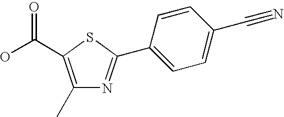

To a suspension of the intermediate 29 (0.5 g, 1.84 mol) in EtOH (30 mL) was added an aqueous LiOH solution (0.12 g, 1.5 eq.) and the mixture was stirred at rt overnight. After evaporation, the resulting mixture was treated with a 1N HCl solution and the resulting precipitate was filtered and dried to give the title compound as a beige solid (0.415 g, 1.7 mmol) in a 92% yield; ¹H NMR (CDCl₃, 300 MHz) δ 8.15 (d, 2H), 7.98 (d, 2H), 2.70 (s, 3H).

### Intermediate 45: 4-Propionyl-benzonitrile

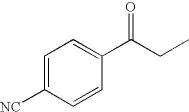

A solution of 1-(4-bromo-phenyl)-propan-1-one (100.0 g, 0.47 mol) and CuCN (48.84 g, 1.1 eq.) in DMF (90 mL) was stirred at 180° C. for 4 hours. The reaction mixture was added to a mixture of FeCl₃ (186.0 g), water (350 mL) and concentrated HCl solution (45 mL). On cooling, the product was extracted with Et₂O, washed with a 1N HCl solution, a 1N NaOH solution and then water, dried over Na₂SO₄, filtered and evaporated. The title compound was obtained as white crystals (51.0 g, 0.32 mol) in a 68.3% yield after purification by distillation under pressure (Teb :180° C.); m.p. 50° C.

### Intermediate 46: 5-(4-Cyano-phenyl)-4-methyl-2H-pyrazole-3-carboxylic acid ethyl ester

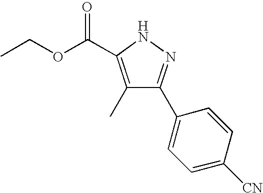

To a solution of LiHMDS in 1M THF solution (100 mL) in cyclohexane (100 mL) was added dropwise a solution of intermediate 45 (15.0 g, 94 mmol) in THF (60 mL). The reaction was stirred at rt for 3 hours. A solution of oxalic acid diethyl ester (15.0 g, 1.05 eq.) in cyclohexane (100 mL) was added and the reaction was stirred at rt overnight. The resulting yellow precipitate was filtered, washed with cyclohexane and dried to give a yellow solid. The solid was dissolved in acetic acid (70 mL) and hydrazine monohydrate was slowly added dropwise (2 mL). The reaction was heated under reflux for 4 hours and poured into water to give a solid. The precipitate was collected by filtration, washed with water, hexane and dried to give a beige solid. Purification by flash chromatography using DCM/MeOH 95/5 as eluent gave the title compound as a white solid (6.0 g, 23 mmol) in a 25% yield; m.p. 148° C.

### Intermediate 47: 5-(4-Cyano-phenyl)4-methyl-2H-pyrazole-3-carboxylic acid

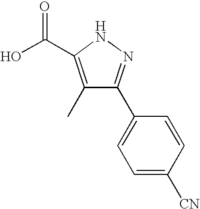

The same method was employed as in the preparation of intermediate 4 but starting from intermediate 46 and gave the title compound as a white solid in 92% yield; m.p. 260° C.

### Intermediate 48: 3-(4-Cyano-phenyl)-4-methyl-pyrazole-1,5-dicarboxylic acid 1-tert-butyl ester 5-ethyl ester

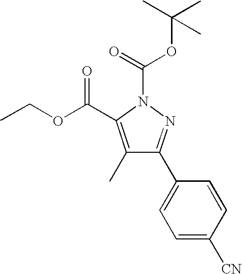

To a solution of intermediate 46 (5.1 g, 20 mmol) in dioxane (80 mL) was added TEA (5 mL) and di-tert-butyl dicarbonate (5.0 g, 1.1 eq.) followed by NaH in 60% oil dispersion (0.5 g) and the suspension was stirred at rt for 4 days. After filtration on a bed of celite, the filtrate was evaporated. The residue was triturated with hot cyclohexane and the resulting precipitate was collected by filtration, washed with cyclohexane and dried. The title compound was obtained as white crystals (4.85 g, 13.7 mol) in a 68.3% yield; m.p. 168° C.

### Intermediate 49: 4-Bromomethyl-3-(4-cyano-phenyl)-pyrazole-1,5-dicarboxylic acid 1-tert-butyl ester 5-ethyl ester

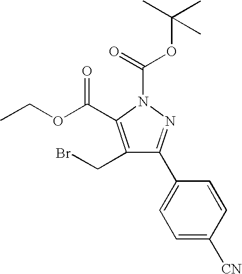

To a solution of intermediate 48 (4.8 g, 13.5 mmol) in CCl₄ (100 mL) was added NBS (2.6 g, 1.1 eq.) and AlBN (60 mg) and the reaction mixture was refluxed overnight. The reaction was cooled and filtered, and the filtrate was concentrated in vaccum to give an oil which was triturated with IprO₂. The title compound was obtained as a white solid (4.6 g, 10.6 mmol) in a 78.4% yield; m.p. 138-140° C.

### Intermediate 50: 5-(4-Cyano-phenyl)-4-hydroxymethyl-2H-pyrazole-3-carboxylic acid ethyl ester

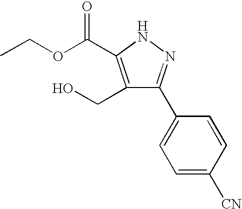

A solution of intermediate 49 (4.3 g, 9.9 mmol) in DMF (25 mL) was treated with a NaOAc solution (3.5 g) in water (15 mL) and the reaction mixture was heated at 120° C. overnight. After concentration in vaccum, the residue was dissolved in water and extracted with DCM to give an oil. Water was added and after neutralisation to pH=6, the residue was triturated and filtered to give a cream solid. The title compound was obtained as white crystals (0.45 g, 1.66 mmol) in a 16.8% yield after purification by flash chromatography using DCM/MeOH 95/5 as eluent; m.p. 165° C.

### Intermediate 51: 5-(4-Cyano-phenyl)-4-hydroxymethyl-2H-pyrazole-3-carboxylic acid

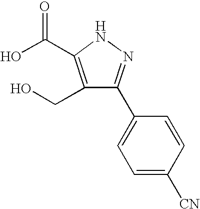

The same method was employed as in the preparation of intermediate 4 but starting from intermediate 50 and gave the title compound as a white solid in a quantitative yield. This intermediate was directly used in the next step.

### Intermediate 52: 2-(4-Cyano-phenyl)4-methyl-oxazole-5-carboxylic acid

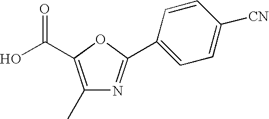

A solution of the available 2-(4-cyano-phenyl)4-methyl-oxazole-5-carboxylic acid methyl ester (1.0 g, 4.13 mmol) in THF (50 mL) was treated with a 1N NaOH solution (4 mL, 0.95 eq.) and the reaction mixture was stirred at rt for 3 days. The mixture was neutralised with a 1N HCl solution and evaporated off. The residue was poured into water to give the title compound as a white solid (0.94 g, 4.13 mmol) in a quantitative yield; LC/MS: M+H C₁₂H₉N₂O₃ 229.

### Intermediate 53: 4-Bromomethyl-2-(4-cyano-phenyl)-oxazole-5-carboxylic acid methyl ester

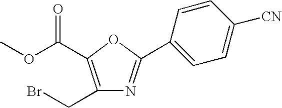

A solution of the available 2-(4-cyano-phenyl)4-methyl-oxazole-5-carboxylic acid methyl ester (2.64 g, 10.9 mmol), NBS (2.33 g, 1.2 eq.) and AlBN (0.27 g, 10% of weight) in CCl₄ (180 mL) was stirred under reflux for 48 hours. After evaporation, the residue was purified by chromatography on silica gel using DCM as eluent. The title compound was obtained as a white solid (2.8 g, 8.72 mmol) in a 80% yield; LC/MS: M+H C₁₃H₁₀BrN₂O₃ 322.

### Intermediate 54: 4-Acetoxymethyl-2-(4-cyano-phenyl)-oxazole-5-carboxylic acid methyl ester

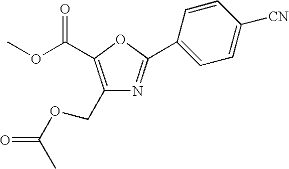

A suspension of intermediate 53 (2.8 g, 8.7 mmol) in acetic acid was treated with NaOAc (3.58 g, 5 eq.) and refluxed for 2 days. After evaporation, water was added and the residue was neutralised. The aqueous layer was extracted with DCM and the organic layer was dried over Na₂SO₄, filtered and evaporated. Purification by flash chromatography using cyclohexane/EtOAc 70/30 and 50/50 as eluent gave the title compound as a white solid (1.05 g, 3.5 mmol) after recrystallisation from cyclohexane; LC/MS: M+H C₁₅H₁₃N₂O₅ 301.

### Intermediate 55: 2-(4-Cyano-phenyl)-4-hydroxymethyl-oxazole-5-carboxylic acid

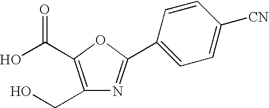

A solution of intermediate 54 (1.05 g, 3.5 mmol) in THF (80 mL) was treated with a 1N solution of NaOH (6.8 mL, 1.95 eq.) and stirred at rt for 35 hours. The mixture was neutralised with 1N HCl solution and the solvent was evaporated. The title compound was obtained as a white solid (0.85 g, 3.5 mmol) and was used directly in the next step without purification; LC/MS: M+H C₁₂H₉N₂O₄ 245.

### Intermediate 56: N-(4-Bromo-phenyl)-methanesulfonamide

To a solution of the available 4-bromo-phenylamine (8.60 g, 50 mmol) and TEA (10.35 g, 2.05 eq.) in DCM (100 mL) cooled to −78° C. was slowly added a solution of methanesulfonyl chloride (6.01 g, 1.05 eq.) in DCM. On warming to rt the reaction mixture was stirred overnight. Water was added and the mixture decantated. The aqueous layer was extracted with DCM and the organic layer was dried over Na₂SO₄, filtered and evaporated. The title compound was obtained as a white solid (6.75 g, 27 mmol) in yield after purification by flash chromatography using DCM as eluent. m.p. 140-142° C.

### Intermediate 57: 4′-Methanesulfonylamino-biphenyl4-carboxylic acid methyl ester

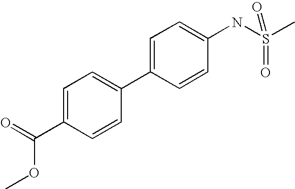

The same method was employed as in the preparation of intermediate 1 but starting from intermediate 56 and the available 4-methoxycarbonylphenylboronic acid and gave the title compound as a pale grey solid in a 33.2% yield after recrystallisation from CH₃CN; m.p. 201-203° C.

### Intermediate 58: 4′-Methanesulfonylamino-biphenyl-4-carboxylic acid

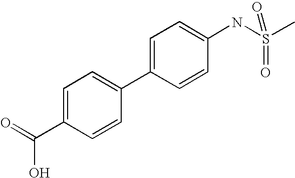

The same method was employed as in the preparation of intermediate 4 but starting from intermediate 57 and gave the title compound as a white solid in a quantitative yield. This intermediate was directly used in the next step without purification; LC/MS: M−H C₁₄H₁₂NO₄S 290.

### Intermediate 59: 4′-Chloro-2-methyl-biphenyl4-carboxylic acid methyl ester

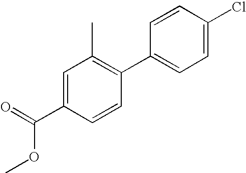

The same method was employed as in the preparation of the intermediate 1 but starting from the available 4-bromo-3-methyl-benzoic acid methyl ester and 4-chlorophenylboronic acid and gave the title compound as a solid in quantitative yield; GC/MS: M⁺ C₁₅H₁₃ClO₂ 260.

### Intermediate 60: 4′-Chloro-2-dibromomethyl-biphenyl-4-carboxylic acid methyl ester

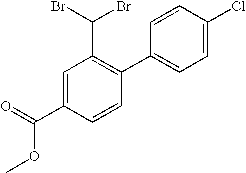

A solution of intermediate 59 (2.0 g, 7.7 mmol), NBS (3.42 g, 2.5 eq.) and AlBN (0.2 g, 10% of weight) in CCl₄ (120 mL) was heated under reflux for 6 hours. After evaporation, the residue was purified by filtration on silica gel using DCM/cyclohexane 50/50 as eluent. The title compound was obtained as a yellow solid (2.8 g, 6.7 mmol) in a 87% yield; 1H NMR (CDCl3, 300 MHz) [symbol] 8.7 (dd, 1H), 7.9 (d, 1H), 7.4 (m, 2H), 7.2 (m, 2H), 7.1 (d, 1H), 6.5 (s, 1H), 3.9 (s, 3H).

### Intermediate 61: 4′-Chloro-2-formyl-biphenyl-4-carboxylic acid methyl ester

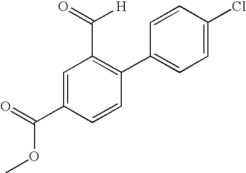

To a suspension of intermediate 60 (0.5 g, 1.2 mmol) in MeOH (50 mL) was added concentrated H₂SO₄ (900 μL, 15 eq.) and the mixture was stirred at rt for 10 min then heated at 60° C. for 2 days. On cooling, the non-reacted starting material was collected by filtration and the filtrate was evaporated. The residue was dissolved in DCM, washed with a NaHCO₃ saturated solution, dried over Na₂SO₄, filtered and evaporated. The title compound was obtained as white crystals (0.035 g, 0.13 mmol) in a 11% yield after purification by filtration on silica gel using DCM as eluent; 1H NMR (CDCl3, 300 MHz) δ 9.9 (s, 1H), 8.6 (s, 1H). 8.2 (d, 1H), 7.4 (m, 3H), 7.25 (d, 2H), 3.9 (s, 3H).

### Intermediate 62: 4′-Chloro-2-dimethylaminomethyl-biphenyl4-carboxylic acid methyl ester

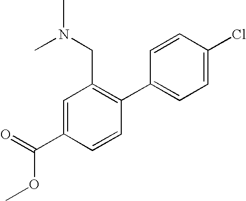

To a solution of intermediate 61 (0.3 g, 1.11 mmol) and dimethylamine (1.2 mL, 2.5 eq.) in a mixture of THF/MeOH/TMOF (15 mL/5 mL/5 mL) was added AcOH (300 μL). After stirring at rt for 2 hours, NaHB(OAc)₃ (0.48 g, 2.0 eq.) was added and the reaction mixture was stirred at rt overnight. After evaporation, the residue was dissolved in DCM, washed with a NaHCO₃ saturated solution, dried over Na₂SO₄, filtered and evaporated. Purification by chromatography on silica gel (DCM/MeOH 90/10) gave the title compound as a yellow oil (0.135 g, 0.45 mmol) in a 40% yield; LC/MS: M+H C₁₇H₁₉ClNO₂ 304.

### Intermediate 63: 4′-Chloro-2-dimethylaminomethyl-biphenyl-4-carboxylic acid

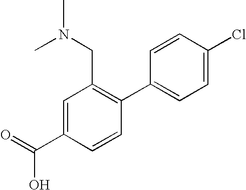

The same method was employed as in the preparation of intermediate 4 but starting from intermediate 62 and gave the title compound as a beige solid in a 50% yield; 1H NMR (CDCl3, 300 MHz) δ 8.2 (s, 1H), 8.05 (d, 1H), 7.6 (m, 4H), 7.5 (d, 1H), 2.6 (s, 2H), 2.2 (s, 6H).

### Intermediate 64: Methyl 4′-[(dimethylamino)carbonyl]-4-biphenylcarboxylate

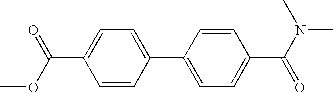

The same method was employed as in the preparation of intermediate 1 but starting from the available 4-methoxycarbonylphenylboronic acid and 4-bromo-N,N-dimethylbenzamide and gave the title compound as a beige solid in a 91% yield; 1H NMR (CDCl3, 300 MHz) δ 8.15 (d, 2H), 7.7 (d, 4H), 7.55 (d, 2H), 3.95 (s, 3H), 3.15 (s, 3H), 3.05 (s, 3H).

### Intermediate 65: 4′-[(Dimethylamino)carbonyl]-4-biphenylcarboxylic acid

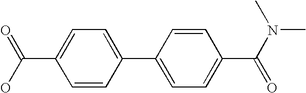

The same method was employed as in the preparation of intermediate 4 but starting from intermediate 64 and gave the title compound as a white solid in a quantitative yield; 1H NMR (DMSO d⁶, 300 MHz) δ 8.15 (d, 2H), 7.75 (dd, 4H), 7.55 (d, 2H), 2.95 (2s, 6H).

### Intermediate 66: (4-{[(4-{4-[1-(Methyloxy)-5,6,7,8-tetrahydro-2-naphthalenyl]-1-piperidinyl}butyl)amino]carbonyl}phenyl)boronic acid

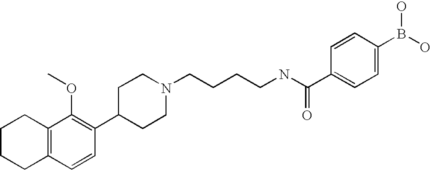

The same method was employed as in the preparation of example 1 but starting from intermediate 19 and the available 4-carboxyphenylboronic acid gave the title compound as a yellow solid in a 62% yield after recrystallisation from CH₃CN; m.p. 139° C.

### Intermediate 67: Ethyl 4-(4-cyanophenyl)-2,4-dioxobutanoate

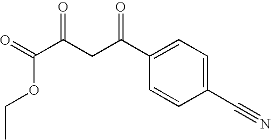

To a solution of the available 4-acetylbenzonitrile (5.0 g, 0.034 mol) in EtOH (100 mL) was added the sodium ethylate (2.8 g, 1.2 eq.) and the reaction was stirred to rt during 1 hour. Then, diethyl oxalate (4.7 mL, 1.0 eq.) was added and the reaction was stirred at rt for 3 hours. After concentration, the mixture was diluted in Et₂O. The formed precipitate was filtered, washed with Et₂O and dried to give the title compound as a pink solid (6.21 g, 0.025 mol) in a 74% yield; LC/MS: M+H C₁₃H₁₂NO₄ 246.

### Intermediate 68: Ethyl 3-(4-cyanophenyl)-1H-pyrazole-5-carboxylate

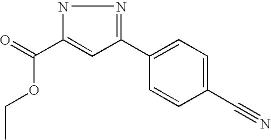

To a solution of intermediate 67 (6.21 g, 0.025 mol) in acetic acid (100 mL) was added dropwise hydrazine hydrate (1.5 mL, 1.2 eq.) and the reaction was refluxed for 24 hours. After cooling, the reaction was triturated with water and ice to give a precipitate which was filtered and dried to give the title compound as a beige solid (3.2 g, 0.013 mol) in a 52.5% yield; LC/MS: M+H C₁₃H₁₂N₃O₂ 242.

### Intermediate 69: Ethyl 3-(4-cyanophenyl)-1-[2-(tetrahydro-2H-pyran-2-yloxy)-ethyl]-1H-pyrazole-5-carboxylate

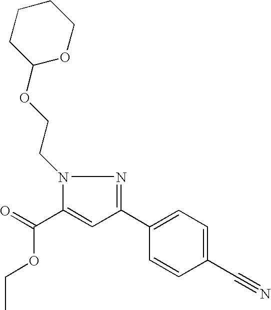

A solution of intermediate 68 (2.0 g, 8.3 mmol) in acetone (200 mL) was stirred for 10 min with cesium carbonate (5.4 g, 2.0 eq.).2-[(2-Bromoethyl)oxy]tetrahydro-2H-pyran (1.50 mL, 1.2 eq.) was added and the reaction mixture was heated under reflux for 3 hours. After cooling, the reaction mixture was concentrated, washed with water and extracted with DCM. The organic layer was dried over Na₂SO₄ and evaporated. After purification by flash chromatography using DCM/MeOH 95/5 as eluent, the title compound was obtained as an oil (2.4 g, 6.5 mol) in a 78.4% yield; LC/MS: M+H C₂₀H₂₄N₃O₄ 370.

### Intermediate 70: 3-(4-Cyanophenyl)-1-[2-(tetrahydro-2H-pyran-2-yloxy)-ethyl]-1H-pyrazole-5-carboxylic acid

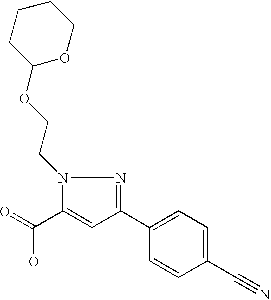

The same method was employed as in the preparation of intermediate 4 but starting from intermediate 69 and gave the title compound as a white solid in a 90%; LC/MS: M−H C₁₈H₁₈N₃O₄ 340.

### Intermediate 71: 5-(4-Cyano-phenyl)-2-[2-(tetrahydro-pyran-2-yloxy)-ethyl]-2H-pyrazole-3-carboxylic acid {4-[4-(1-methoxy-5,6,7,8-tetrahydro-naphthalen-2-yl)-piperidin-1-yl]-1-butyl}-amide

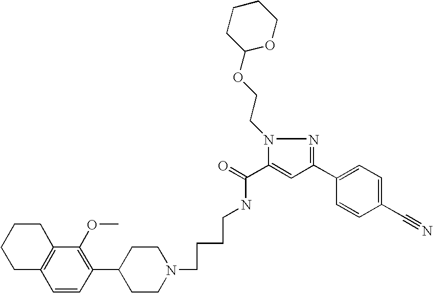

The same method was employed as in the preparation of example 1 but starting from intermediate 19 and intermediate 70 and gave the title compound as a white solid after purification by flash chromatography using DCM/MeOH 90/10 as eluent; LC/MS: M+H C₃₈H₅₀N₅O₄ 640.

### Intermediate 72: 4′-Acetylamino-biphenyl-4-carboxylic acid {4-[4-(1-methoxy-5,6,7,8-tetrahydro-naphthalen-2-yl)piperidin-1-yl]-butyl}-amide

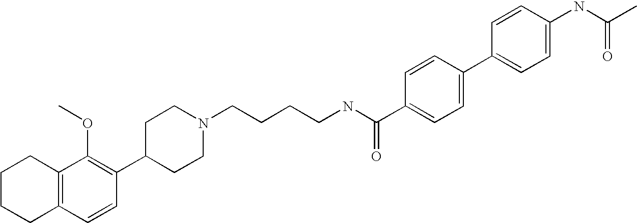

The same method was employed as in the preparation of intermediate 1 but starting from intermediate 66 and the available 4′-bromoacetanilide and gave the title compound as a white solid in a 28% yield after crystallisation from CH₃CN; m.p. 239-241° C.

### Intermediate 73: 4′-Amino-biphenyl-4-carboxylic acid {4-[4-(1-methoxy-5,6,7,8-tetrahydro-naphthalen-2-yl)-piperidin-1-yl]-butyl}amide

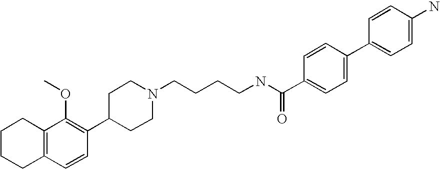

A solution of intermediate 72 (0.47 g, 0.85 mmol) in 1N HCl solution (11 mL) was heated under reflux for 16 hours. The reaction was treated with 1N NaOH solution (pH=12) and the precipitate was filtered, washed with water and dried to give the title compound as a beige solid (0.37 g, 0.72 mmol) in a 86% yield; LC/MS: M+H C₃₃H₄₂N₃O₂ 512.

### Intermediate 74: N-(4-Bromophenyl)-2-propanesulfonamide

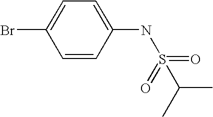

To a solution of the available 4-bromoaniline (1.04 g, 6.07 mmol) in DCM (20 mL) was added TEA (1.35 g, 2.2 eq.) and 2-propanesulfonyl chloride (1.04 g, 1.2 eq.) and the reaction mixture was stirred at rt for 24 hours. The mixture was washed with water, the organic layer was dried over Na₂SO₄ and evaporated to give the title compound as a white solid (0.3 g, 1 mmol) after purification by flash chromatography using DCM and DCM/MeOH 90/10 as eluents in a 17.9% yield; m.p. 98-100° C.

### Intermediate 75: Ethyl 3-hydroxy-4-iodobenzoate

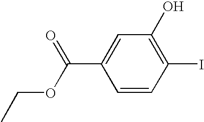

A solution of the available 3-hydroxy-4-iodobenzoic acid (25.0 g, 94.7 mmol) in EtOH (1000 mL) was treated with a HCl gas for 30 min and the reaction was heated under reflux for 48 hours. On cooling the mixture was evaporated and residue was purified with silica using DCM/MeOH 97/3 as eluent. The title compound was obtained as a white solid (25.66 g, 94.7 mmol) in a 93% yield; ¹H NMR (CDCl₃, 300 MHz) δ 7.75 (d, 1H), 7.5 (d, 1H), 7.45 (dd, 1H), 4.5 (q, 2H), 1.4 (t, 3H).

### Intermediate 76: Ethyl 4′-chloro-2-hydroxy-4-biphenylcarboxylate

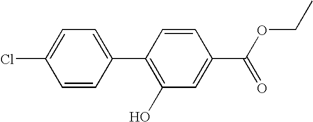

The same method was employed as in the preparation of intermediate 1 but starting from intermediate 75 and the available 4-chlorocarbonylphenylboronic acid and gave the title compound as a white solid in 74% yield after purification by flash chromatography using DCM and DCM/AcOEt 97/3 as eluents; LC/MS: M+H C₁₅H₁₄ClO₃ 277.

### Intermediate 77: 4′-Chloro-2-hydroxy-4-biphenylcarboxylic acid

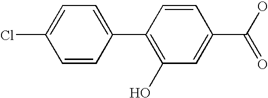

The same method was employed as in the preparation of intermediate 4 but starting from intermediate 76 and gave the title compound as a white solid in a quantitative yield; LC/MS: M+H C₁₃H₁₀ClO₃ 249.

### Intermediate 78: 5-Bromo-2-thiophenecarbonitrile

To a solution of the available 2-thiophenecarbonitrile (2.22 g, 20.3 mmol) in a mixture of acetic acid (1.22 g, 20.3 mmol) and acetic anhydride (8.3 g, 81.3 mmol) was added NBS (3.62 g, 1.0 eq.) and bromine (1.05 mL, 1.0 eq.) and the reaction mixture was stirred at rt for 3 hours. Water and ice were added and also a saturated sodium bisulfite solution. After extraction with DCM, the organic layer was washed with a saturated NaHCO₃ solution, dried over Na₂SO₄ and evaporated. The title compound was obtained as a orange liquid (3.74 g, 20 mmol) in a quantitative yield; GC/MS: M⁺ C₅H₂BrNS 188.

### Intermediate 79: 5-Bromo-2-chloropyridine

To a solution of the available 5-amino-2-chloropyridine (3.0 g, 23.3 mmol) in HBr solution 48% at −10° C. was added a sodium nitrite solution (4.18 g, 60.7 mmol) in water (6 mL). Bromine (11.2 g, 3 eq.) was slowly added and the reaction was stirred at rt for 48 hours. After addition of a NaOH solution (16.8 g) in water (42 mL), and extraction with Et₂O, the organic layer was dried over Na₂SO₄ and evaporated. After purification by flash chromatography using DCM/Cyclohexane 50/50 as eluent, the title compound was obtained as:a white solid (0.135 g, 0.7 mmol) in 3% yield; GC/MS: M⁺ C₅H₃BrClN 192.

### Intermediate 80: 3-Bromo-6-chloropyridazine

The same method was employed as in the preparation of intermediate 79 but starting from the available 6-chloro-3-pyridazinamine and gave the title compound as a white solid in 43.5% yield after purification by flash chromatography using DCM as eluent; GC/MS: M⁺ C₄H₂BrClN₂ 193.

### Intermediate 81: Methyl 5-(4-chlorophenyl)-2-pyridinecarboxylate

The same method was employed as in the preparation of intermediate 1 but starting from the available methyl 5-bromo-2-pyridinecarboxylate and 4-chlorocarbonylphenylboronic acid and gave the crude title compound which was directly used in the next step.

### Intermediate 82: 5-(4-Chlorophenyl)-2-pyridinecarboxylic acid

The same method was employed as in the preparation of intermediate 4 but starting from intermediate 81 and gave the title compound as a white solid in a 53% yield; m.p.>260° C.; LC/MS: M−H C₁₂H₇ClNO₂ 232.

### Intermediate 83: Methyl 6-(4-chlorophenyl)-3-pyridinecarboxylate

The same method was employed as in the preparation of intermediate 1 but starting from the available Methyl 6-chloro-3-pyridinecarboxylate and 4-chlorocarbonylphenylboronic acid gave the title compound as a white solid in a 90% yield after purification by flash chromatography using DCM as eluent; m.p. 124-126° C.

### Intermediate 84: 6-(4-Chlorophenyl)-3-pyridinecarboxylic acid

The same method was employed as in the preparation of intermediate 4 but starting from intermediate 83 gave the title compound as a white solid in a quantitative yield; m.p.>260° C.; LC/MS: M+H C₁₂H₉ClNO₂ 234.

### Intermediate 85: Methyl 6-(4-cyanophenyl)-3-pyridinecarboxylate

The same method was employed as in the preparation of intermediate 1 but starting from the available methyl 6-chloro-3-pyridinecarboxylate and 4-cyanocarbonylphenylboronic acid and gave the title compound as a white solid in a 32% yield after purification by flash chromatography using DCM and DCM/AcOEt 98/2 as eluent; m.p. 142-144° C.

### Intermediate 86: 6-(4-Cyanophenyl)-3-pyridinecarboxylic acid

The same method was employed as in the preparation of intermediate 4 but starting from intermediate 85 and gave the title compound as a white solid in a 91% yield; m.p.>260° C.; LC/MS: M+H C₁₂H₉N₂O₂ 225.

### Intermediate 87: Methyl 6-(5-chloro-2-thienyl)-3-pyridinecarboxylate

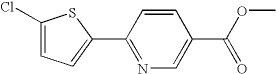

The same method was employed as in the preparation of intermediate 1 but starting from the available methyl 6-chloro-3-pyridinecarboxylate and 5-chlorothiophene-2-boronic acid and gave the title compound as a yellow solid in a 26% yield after purification by flash chromatography using DCM as eluent; m.p. 122-124° C.

### Intermediate 88: 6-(5-Chloro-2-thienyl)-3-pyridinecarboxylic acid

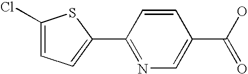

The same method was employed as in the preparation of intermediate 4 but starting from intermediate 87 and gave the title compound as a yellow solid in a 61% yield; m.p.>260° C.; LC/MS M+H C₁₀H₇ClNO₂S 240.

### Intermediate 89: 5-(4-Cyanophenyl)-2-pyridinecarboxylic acid

The same method was employed as in the preparation of intermediate 1 but starting from the available methyl 5-bromo-2-pyridinecarboxylate and 4-cyanocarbonylphenylboronic acid and gave the title compound (carboxylic acid form) as a white solid in a quantitative yield; m.p.>260° C.; LC/MS: M−H C₁₃H₇N₂O₂ 223.

### Intermediate 90: Methyl 6-(5-cyano-2-thienyl)-3-pyridinecarboxylate

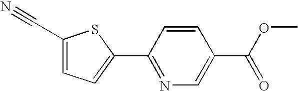

The same method was employed as in the preparation of intermediate 1 but starting from the available methyl 6-chloro-3-pyridinecarboxylate and 5-cyanothiophene-2-boronic acid and gave the title compound as a yellow solid in a 6.8% yield after recrystallisation from AcOEt; m.p.164-166° C.

### Intermediate 91: 6-(5-Cyano-2-thienyl)-3-pyridinecarboxylic acid

The same method was employed as in the preparation of intermediate 4 but starting from intermediate 90 and gave the title compound as a yellow solid in a 48.5% yield; LC/MS: M+H C₁₁H₇N₂O₂S 231.

### Intermediate 92: 5-Bromo-2-pyridinecarbonitrile

The same method was employed as in the preparation of intermediate 79 but starting from 5-amino-2-pyridinecarbonitrile and gave the title compound as a white solid in a 10% yield after purification by flash chromatography using DCM as eluent; m.p. 130-132° C.

### Intermediate 93: ethyl 1,4-dimethyl-1H-imidazole-5-carboxylate

To a solution of the available ethyl 4-methyl-1H-imidazole-5-carboxylate (5.0 g, 32.5 mmol) in DMF (50 mL) was added NaHCO₃ (5.72 g, 2.1 eq.) and methyl iodide (2.43 mL, 1.2 eq.). The reaction was stirred at 90° C. during 5 days. After evaporation of the solvant, the residue was diluted in DCM and washed with water. The organic layer was dried over Na₂SO₄ and evaporated off. After purification by flash chromatography using DCM as eluent, the title compound was obtained as a yellow oil (1.69 g, 0.01 mol) in 31% yield; ¹H NMR (CDCl₃, 300 MHz) δ 7.86 (s, 1H), 4.36 (q, 2H), 3.89 (s, 3H), 2.47 (s, 3H), 1.4 (t, 3H).

### Intermediate 94: ethyl 2-bromo-1,4-dimethyl-1H-imidazole-5-carboxylate

To a solution of intermediate 93 (1.69 g. 0.01 mol) in CH₃CN (60 mL) was added NBS (2.15 g, 1.2 eq.) and the reaction was stirred at rt for 1 night. After evaporation of the solvant, the residue was dissolved in DCM and washed with water. The organic layer was dried over Na₂SO₄ and evaporated off. After purification by flash chromatography using DCM/MeOH 90/10 as eluent, the title compound was obtained as yellow crystals (0.645 g, 2.6 mmol) in 26% yield; ¹H NMR (CDCl₃, 300 MHz) δ 4.55 (q, 2H), 4.09 (s, 3H), 2.69 (s, 3H), 1.61 (t, 3H).

### Intermediate 95: ethyl 2-(4-chlorophenyl)-1,4-dimethyl-1H-imidazole-5-carboxylate

The same method was employed as in the preparation of intermediate 1 but starting from intermediate 94 and the available 4-chlorocarbonylphenylboronic acid and gave the title compound as a white solid in a 50% yield after purification by flash chromatography using DCM and DCM/MeOH 99/1as eluents; LC/MS: M+H C₁₄H₁₅N₂O₂Cl 279.

### Intermediate 96: 2-(4-chlorophenyl)-1,4-dimethyl-1H-imidazole-5-carboxylic acid

The same method was employed as in the preparation of intermediate 4 but starting from intermediate 95 and gave the title compound as a white solid in a 95% yield; LC/MS: M+H C₁₂H₁₁ClN₂O₂ 251.

### EXAMPLES

### Example 1

### 2-Hydroxymethyl-4′-trifluoromethyl-biphenyl-4-carboxylic acid {4-[4-(1H-indol-3-yl)-piperidin-1-yl]-butyl}-amide

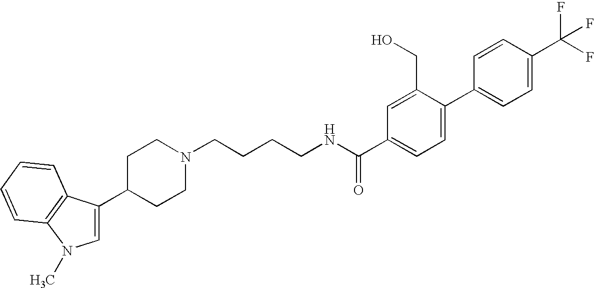

A solution of intermediate 4 (2.0 g, 7 mmol ), intermediate 8 (2.0 g, 1.0 eq.), EDCl (2.02 g, 1.5 eq.), HOBT (1.43 g, 1.5 eq.) and TEA (1.5 mL, 1.5 eq.) in DMF was stirred for 24 hours at rt. The solvent was removed, the residue diluted with DCM and washed with water and with a 1N NaOH solution. The organic layer was dried over Na₂SO₄ and the solvent evaporated. After purification by flash chromatography using DCM/MeOH (95/5) as eluent, the title compound (2.3 g, 4 mmol) was obtained as white crystals in 58% yield after crystallisation from acetonitrile; m.p. 177° C.; LC/T of: ES⁺ 564.2867 5.3 ppm.

### Example 2

### 4-Hydroxymethyl-2-(4-trifluoromethyl-phenyl)-thiazole-5-carboxylic acid {4-[4-(1-methoxy-5,6,7,8-tetrahydro-naphthalen-2-yl)-piperidin-1-yl]-butyl}-amide

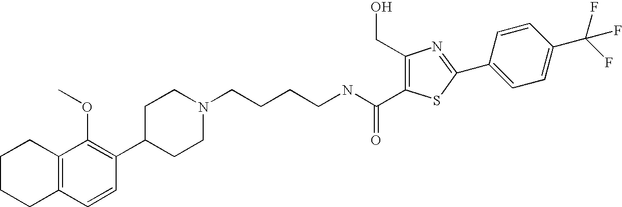

The same method was employed as in the preparation of example 1 but starting from intermediate 13 and intermediate 19 and gave the title compound as white crystals in a 36% yield after recrystallisation in. EtOH; m.p. 179-180° C; ¹H NMR (CDCl₃, 300 MHz) [symbol] 8.6 (s, 1H), 8.0 (d, 2H), 7.6 (d, 2H), 6.9 (d, 1H), 6.7 (d, 2H), 4.9 (s, 2H), 3.6 (s, 3H), 3.4 (m, 2H), 3.0 (m, 2H), 2.8 (m, 1H), 2.6 (m, 4H), 2.3 (m, 2H), 1.6 (m, 12H).

### Example 3

### 4-Hydroxymethyl-2-(4-trifluoromethyl-Phenyl)-thiazole-5-carboxylic acid {4-[4-(1-methyl-1H-indol-3-yl)-piperidin-1-yl]-butyl}-amide

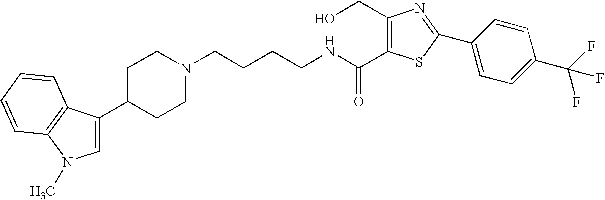

The same method was employed as in the preparation of example 1 but starting from intermediate 13 and intermediate 8 and gave the title compound as a yellow powder in a 38% yield after recrystallisation in CH₃CN; m.p. 160° C.; LC/T of: ES⁺ 571.2358 0.5 ppm.

### Example 4

### 4-Hydroxymethyl-2-(4-trifluoromethyl-phenyl)-thiazole-5-carboxylic acid {4-[4-(2-ethoxy-4-ethyl-phenyl)-piperidin-1-yl]-butyl}-amide

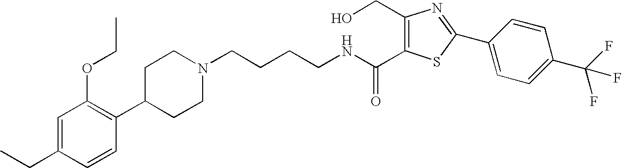

The same method was employed as in the preparation of example 1 but starting from intermediate 13 and intermediate 25 and gave the title compound as beige crystals in a 25% yield after recrystallisation in CH₃CN; m.p. 150° C.; LC/MS: M+H C₃₁H₃₉F₃N₃O₃S 590.

### Example 5

### 4-Hydroxymethyl-2-(4-trifluoromethyl-phenyl)-thiazole-5-carboxylic acid {4-[4-(1H-indol-3-yl)-piperidin-1-yl]-butyl}-amide

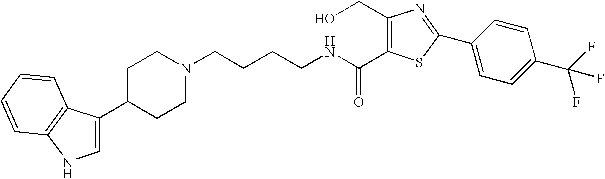

The same method was employed as in the preparation of example 1 but starting from intermediate 27 and intermediate 13 and gave the title compound as yellow crystals in 31% yield after recrystallisation in CH₃CN/EtOH; m.p. 235° C.; LC/T of: ES⁺ 557.2158 7.1 ppm.

### Example 6

### 3-Hydroxymethyl-4′-trifluoromethyl-biphenyl4-carboxylic acid {4-[4-(1H-indol-3-yl)-piperidin-1-yl]-butyl}-amide

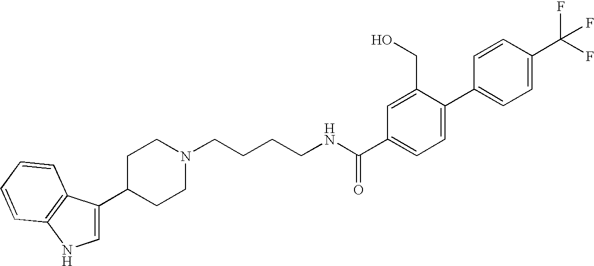

The same method was employed ;as in the preparation of example 1 but starting from intermediate 27 and intermediate 4 and gave the title compound as white crystals in a 42% yield after crystallisation in DMF; m.p. 255° C.;LC/T of: ES⁺ 550.2725 8.0 ppm.

### Example 7

### 2-(4-Cyano-phenyl)-hydroxymethyl-thiazole-5-carboxylic acid {4-[4-(1-methoxy-5,6,7,8-tetrahydro-naphthalen-2-yl)-piperidin-1-yl]-butyl}-amide

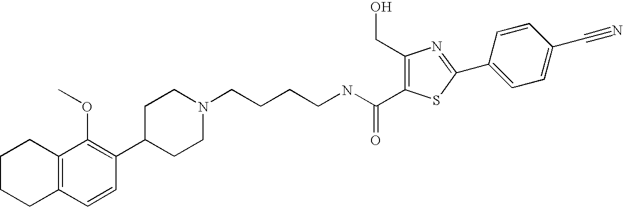

The same method was employed as in the preparation of example 1 but starting from intermediate 32 and intermediate 19 and gave the title compound as beige crystals in a 8% yield after crystallisation in ethyl acetate; m.p. 212° C.; LC/MS: M−H C₃₂H₃₇N₄O₃S 557.

### Example 8

### 4-{4-[4-(1-Methoxy-5,6,7,8-tetrahydro-naphthalen-2-yl)-piperidin-1-yl]-butylcarbamoyl}-4′-trifluoromethyl-biphenyl-2-carboxylic acid methyl ester

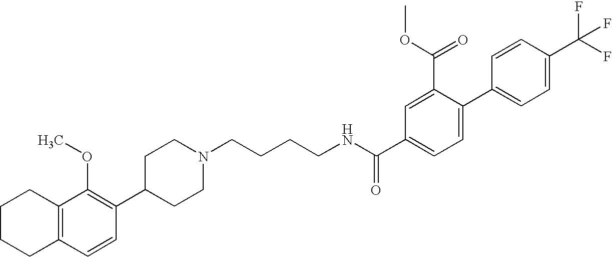

The same method was employed as in the preparation of example 1 but starting from intermediate 35 and intermediate 19 and gave the title compound as white crystals in a 51% yield after crystallisation from iPrO₂.; m.p. 132° C.; LC/T of: ES⁺ 623.3063 5.3 ppm.

### Example 9

### 2-Hydroxymethyl-4-trifluoromethyl-biphenyl-4-carboxylic acid }4-[4-(1-isopropyl-5-methoxy-1H-indol-3-yl)-piperidin-1-yl]-butyl}-amide

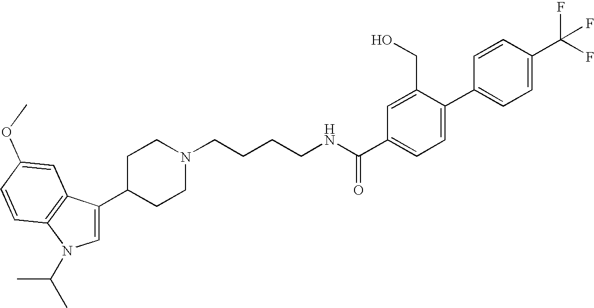

The same method was employed as in the preparation of example 1 but starting from intermediate 40 and intermediate 4 and gave the title compound as a white solid in a 39% yield after crystallisation in hexane; m.p. 90-100° C.; ¹H NMR (CDCl₃, 300 MHz) [symbol] 8.02 (s, 1H), 7.76 (d, 1H), 7.60 (d, 2H), 7.38 (d, 2H), 7.25-7.14 (m, 2H), 7.02 (s, 1H), 6.86 (s, 2H), 4.53 (s, 2H) 3.83 (s, 3H), 3.47 (m, 2H), 3.02 (m, 2H), 2.75 (m, 1H), 2.41 (m, 2H), 2.13-1.97 (m, 4H), 1.67 (m, 4H), 1.41 (d, 6H).

### Example 10

### 2-(4-Chloro-phenyl)-4-hydroxymethyl-thiazole-5-carboxylic acid {4-[4-(1-methoxy-5,6,7,8-tetrahydro-naphthalen-2-yl)-piperidin-1-yl]-butyl}-amide

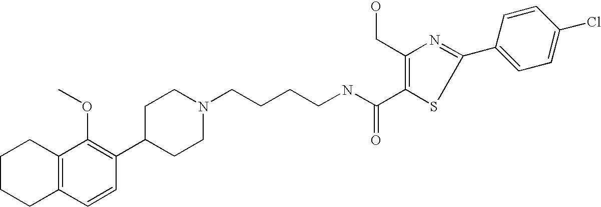

The same method was employed as in the preparation of example 1 but starting from intermediate 43 and intermediate 19 and gave the title compound as yellow solid in a 21% yield after recrystallisation from EtOH; m.p. 198-200° C.; LC-T of: ES+ Calculated, 570.2557 Found, 570.2502 -9.6 ppm.

### Example 11

### 2-(4-Cyano-phenyl)4-methyl-thiazole-5-carboxylic acid {4-[4-(1-methoxy-5,6,7,8-tetrahydro-naphthalen-2-yl)-piperidin-1-yl]-butyl}-amide

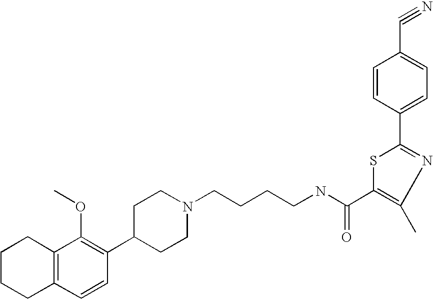

The same method was employed as in the preparation of example 1 but starting from intermediate 44 and intermediate 19 and gave the title compound as a beige solid in 44% yield after crystallisation from IprO₂; m.p. 160-164° C.; LC-T of: ES+ calculated 543 ;2794; Found, 543.2834 7.3 ppm.

### Example 12

### 2-(4-Chloro-phenyl)4-methyl-thiazole-5-carboxylic acid {4-[4-(1-methoxy-5,6,7,8-tetrahydro-naphthalen-2-yl)-piperidin-1-yl]-butyl}-amide

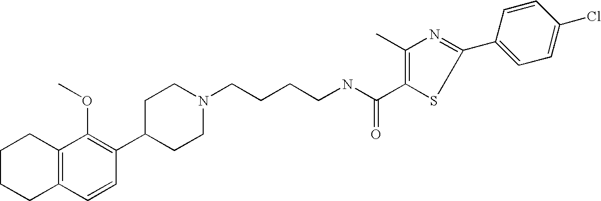

The same method was employed s in the preparation of example 1 but starting from intermediate 19 and the available 2-(4-chloro-phenyl)-4-methyl-thiazole-5-carboxylic acid and gave the title compound as a beige solid (473 mg, 0.86 mmol) in a 38.5% yield after purification by flash chromatography using DCM/MeOH 90/10 as eluent, m.p. 151° C.; LC/MS: M+H C₃₁H₃₉ClN₃O₂S 552.

### Example 13

### 5-(4-Cyano-phenyl)-4methyl-2H-pyrazole-3-carboxylic acid {4-[4-(1-methoxy-5,6,7,8-tetrahydro-naphthalen-2-yl)-piperidin-1-yl]-butyl}-amide

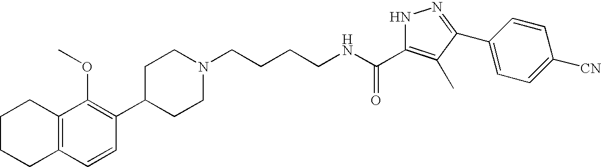

The same method was employed as in the preparation of example 1 but starting from intermediate 47 and intermediate 19 and gave the title compound as white crystals in a 34.6% yield after recrystallisation from CH₃CN; m.p. 222° C.; LC-T of: ES+ Calculated, 526.3182 Found, 526.3207 4.8 ppm.

### Example 14

### 5-(4-Cyano-phenyl)-4-hydroxymethyl-2H-pyrazole-3-carboxylic acid }4-[4-(1-methoxy-5,6,7,8-tetrahydro-naphthalen-2-yl)-piperidin-1-yl]-butyl}-amide

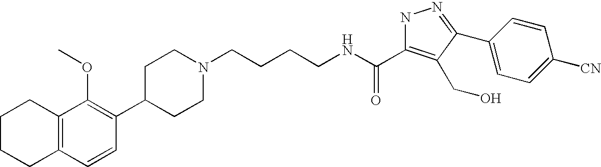

The same method was employed as in the preparation of example 1 but starting from intermediate 51 and intermediate 19 and gave the title compound as a solid in 5.6% yield after crystallisation from CH₃CN; m.p. 185-186° C.; LC/MS: M+H C₃₂H₄₀N₅O₃ 542.

### Example 15

### 2-(4-Cyano-phenyl)-4-methyl-oxazole-5-carboxylic acid {4-[4-(1-methoxy-5,6,7,8-tetrahydro-naphthalen-2-yl)-piperidin-1-yl]-butyl}amide

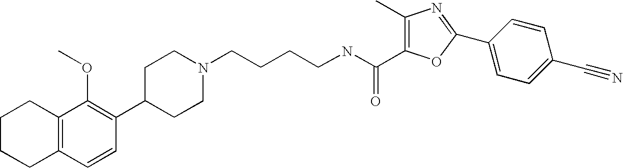

The same method was employed as in the preparation of example 1 but starting from intermediate 52 and intermediate 19 and gave the title compound as a white solid in a 51% yield after crystallisation from IPrO₂; m.p. 157° C.; LC/MS M+H C₃₂H₃₉N₄O₃ 527.

### Example 16 2-(4-Cyano-phenyl)-4-hydroxymethyl-oxazole-5-carboxylic acid {4-[4-(1-methoxy-5,6,7,8-tetrahydro-naphthalen-2-yl)-piperidin-1-yl]-butyl}-amide

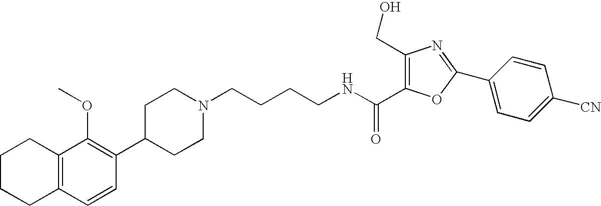

The same method was employed as in the preparation of example 1 but starting from intermediate 55 and intermediate 19 and gave the title compound as white solid in a 25% yield after crystallisation frorn IPrO₂; m.p. 160-162° C.; LC/MS: M+H C₃₂H₃₉N₄O₄ 543.

### Example 17

### 4′-Methanesulfonylamino-biphenyl-4-carboxylic acid {4-[4-(1-methoxy-5,6,7,8-tetrahydro-naphthalen-2-yl)-piperidin-1-yl]-butyl}-amide

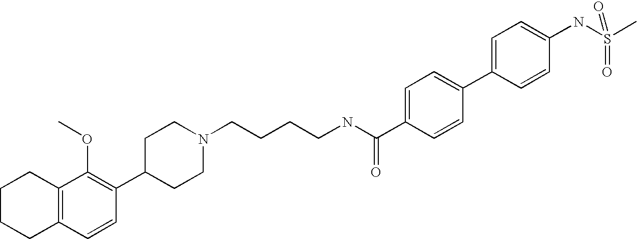

The same method was employed as in the preparation of example 1 but starting from intermediate 58 and intermediate 19 and gave the title compound as a white solid in a 48% yield after crystallisation from CH₃CN; m.p. 200-202° C.; LC-T of: ES+ Calculated, 590.3052 ; Found, 590.2764 12.0 ppm.

### Example 18

### 2-Dimethylaminomethyl-biphenyl4-carboxylic acid {4-[4-(1-methoxy-5,6,7,8-tetrahydro-naphthalen-2-yl)-piperidin-1-yl]-butyl}-amide

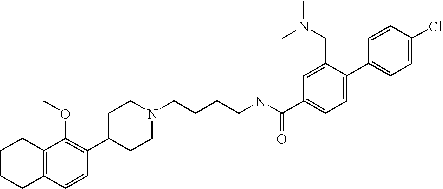

The same method was employed as in the preparation of example 1 but starting from intermediate 63 and intermediate 19 and gave the title compound as a yellow oil in a 12% yield after purification by flash chromatography using DCM/MeOH 85/15 as eluent; LC-T of: ES+ Calculated 588.3357; Found, 588.3286 −12.0 ppm.

### Example 19

### Biphenyl-4,4′-dicarboxylic acid 4-dimethylamide 4′-({4-[4-(1-methoxy-5,6,7,8-tetrahydro-naphthalen-2-yl)-piperidin-1-yl]-butyl}-amide

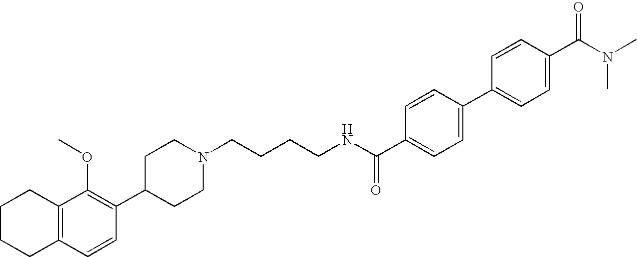

The same method was employed as in the preparation of example 1 but starting from intermediate 65 and intermediate 19 and gave the title compound as a white solid in 59% yield after purification by flash; chromatography using DCM/MeOH 90/10 as eluent and crystallisation from IprO₂; m.p. 170° C.; LC/MS: M+H C₃₆H₄₆N₃O₃ 568.

### Example 20

### 4-(5-Acetyl-thiophen-2-yl)-N-{4-[4-(1-methoxy-5,6,7,8-tetrahydro-naphthalen-2-yl)-piperidin-1-yl]-butyl}-benzamide

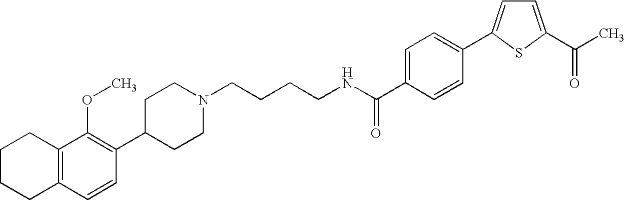

The same method was employed as in the preparation of the intermediate 1 but starting from the intermediate 66 and the available 2-Acetyl-5-bromothiophene gave the title compound as a beige solid in a 28.6% yield after crystallisation from CH₃CN; m.p. 216-218° C.; LC/MS: M+H C₃₃H₄₁N₂O₃S 545.

### Example 21

### 5-(4-Cyano-phenyl)-2-(2-hydroxy-ethyl)-2H-pyrazole-3-carboxylic acid {4-[4-(1-methoxy-5,6,7,8-tetrahydro-naphthalen-2-yl)-piperidin-1-yl]-butyl}-amide

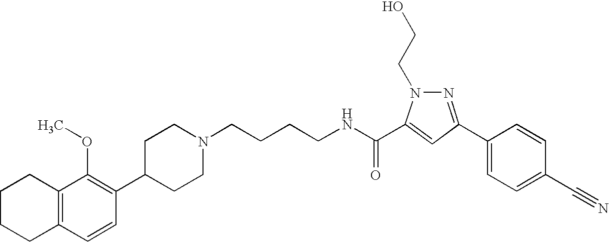

Intermediate 71 (1.21 g, 1.9 mmol) in THF (50 mL) was stirred with a 1N HCl solution for 48 hours. The resulting precipitate was filtered, washed with water and dried to give the title compound (as chlorhydrate salt form) in a 89.3% yield; m.p. 152° C.; LC-T of: ES+ Calculated, 556:3287 Found, 556.3347 10.90 ppm.

### Example 22

### 4′-(2,2,2-Trifluoro-ethanesulfonylamino)-biphenyl4-carboxylic acid {4-[4-(1-methoxy 5,6,7,8-tetrahydro-naphthalen-2- yl)-piperidin-1-yl]-butyl}-amide

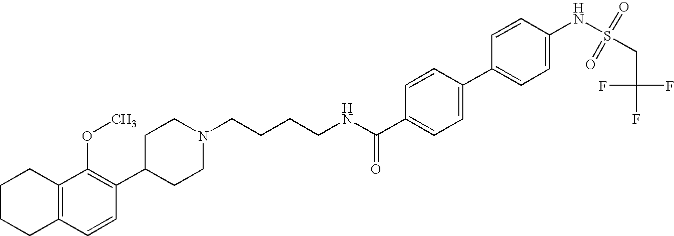

To a solution of intermediate 73 (0 2 g, 0.39 mmol) in DCM (10 mL) was added TEA (91 mg, 2.3 eq) and the available 2,2,2-trifluoroethanesulfonyl chloride (0.092 g, 1.3 eq.) and the reaction mixture was stirred at rt for 16 hours. The mixture was washed with a NaHCO₃ saturated solution: and brine. The organic layer was dried over Na₂SO₄ and evaporated to give the title, compound as a beige solid ( 0.056 g, 0.085 mmol) in a 22% yield after recrystallisation from CH₃CN; m.p. 182-184° C.; LC/MS: M+H C₃₅H₄₃F₃N₃O₄S 658.

### Example 23

### 4-(5-Chloro-thiophen-2-yl)-N-{4-[4-(1-methoxy-5,6,7,8-tetrahydro-naphthalen-2-yl)-piperidin-1-yl]-butyl}-benzamide

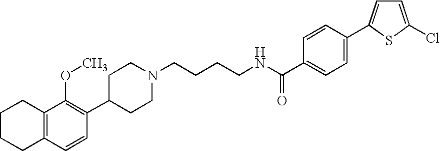

The same method was employed as in the preparation of intermediate 1 but starting from intermediate 66 and the available 2-bromo-5-chlorothiophene and gave the title compound as a beige solid after crystallisation from CH₃CN; m.p. 185-187° C.; LC/MS: M+H C₃₁H₃₈ClN₂O₂S 537.

### Example 24

### 4′-(Propane-2-sulfonylamino)-biphenyl-4-carboxylic acid }4-[4-(1-methoxy-5,6,7,8-tetrahydro-naphthalen-2-yl)-piperidin-1-yl]-butyl}-amide

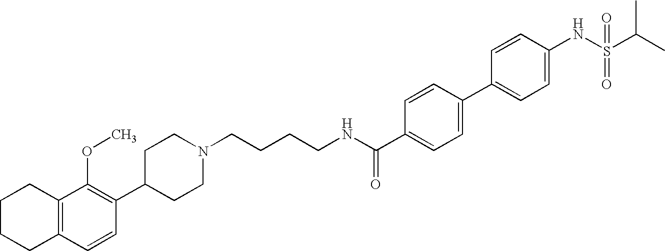

The same method was employed as in the preparation of intermediate 1 but starting from intermediate 66 and intermediate 74 and gave the title compound as a beige solid in 5.6% yield after crystallization from CH₃CN, m.p. 188-190° C.; LC/MS: M+H C₃₆H₄₈N₃O₄S 618.

### Example 25

### 4′-Chloro-2-hydroxy-biphenyl-4-carboxylic acid {4-[4-(1-methoxy-5,6,7,8-tetrahydro-naphthalen-2-yl-piperidin-1-yl]-butyl}-amide

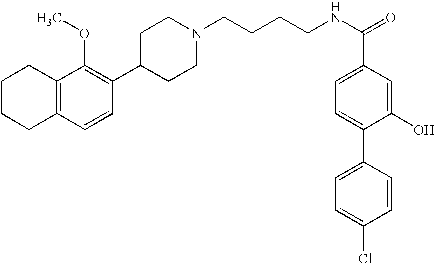

The same method was employed as in the preparation of example 1 but starting from intermediate 77 and intermediate 19 and gave the title compound as a white solid in a 90% yield after crystallisation from IprO₂; m.p.199° C.; LC/MS: M+H C₃₃H₄₀ClN₂O₃ 547.

### Example 26

### 4′-(2,2,2-Trifluoro-acetyl)-biphenyl-4-carboxylic acid {4-[4-(1-methoxy-5,6,7,8-tetrahydro-naphthalen-2-yl)-piperidin-1-yl]-butyl}-amide

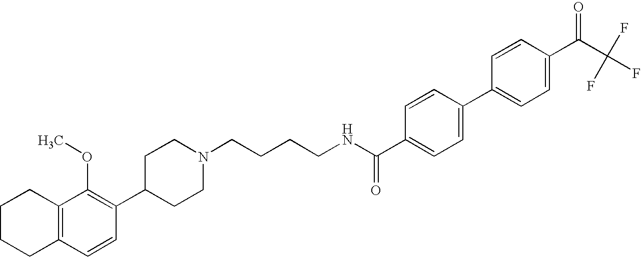

The same method was employed as in the preparation of the intermediate 1 but starting from the intermediate 66 and the available 4′-Bromo-2,2,2-trifluoroacetophenone gave the title compound as a white solid in a 10.1% yield after recrystallisation from CH₃CN; m.p. 197-199° C.; LC/MS: M+H C₃₅H₄₀F₃N₂O₃ 593.

### Example 27

### 4-(5-Cyano-thiophen-2-yl)-N-{4-[4-(1-methoxy-5,6,7,8-tetrahydro-naphthalen-2-yl)-piperidin-1-yl]-butyl}-benzamide

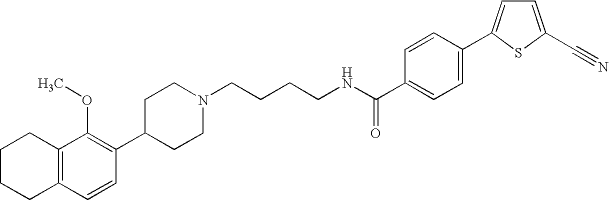

The same method was employed as in the preparation of intermediate 1 but starting from intermediate 66 and intermediate 78 and gave the title compound as a beige solid in 26.5% yield after purification by flash chromatography using DCM/MeOH 90/10 as eluent; m.p. 182-184° C.; LC-T of: ES+ Calculated, 528.2684; Found, 528.2640 −8.3 ppm.

### Example 28

### 2-Carbamoylmethoxy-4′-chloro-biphenyl-4-carboxylic acid {4-[4-(1-methoxy-5,6,7,8-tetrahydro-naphthalen-2-yl)-piperidin-1-yl]-butyl}-amide

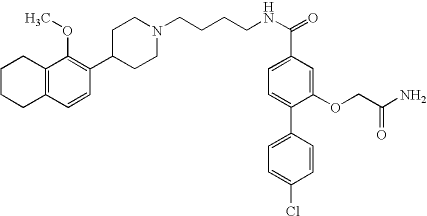

To a solution of example 25 (0.2 g, 0.37 mmol) in DMF (30 mL) was added cesium carbonate (0.3 g, 2.5 eq.) and the available 2-bromoacetamide (0.07 g, 1.3 eq.) and the reaction was stirred at 70° C. for 48 hours. After evaporation, the residue was diluted in DCM and washed with water. The organic layer was dried over Na₂SO₄ and evaporated. The title compound was obtained as a white solid in 72% yield after crystallisation from IprO₂; m.p. 198° C.; LC-T of: ES+ Calculated, 604.2942; Found, 604.2897 −7.5 ppm.

### Example 29

### 4′-(2,2,2-Trifluoro-acetylamino)-biphenyl-4-carboxylic acid {4-[4-(1-methoxy-5,6,7,8-tetrahydro-naphthalen-2-yl)-piperidin-1-yl]-butyl}-amide

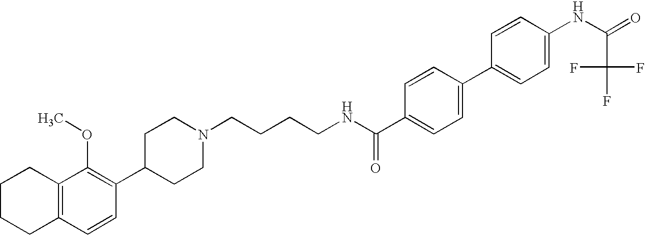

The same method was employed as in the preparation of example 22 but starting from intermediate 73 and trifluoroacetic anhydride and gave the title compound as a beige solid in 25.3% yield after recrystallisation from CH₃CN/MeOH 1/1; m.p. 226-228° C.; LC/MS: M+H C₃₅H₄₁F₃N₃O₃ 608.

### Example 30

### 4-(5-Cyano-pyridin-2-yl)-N-{4-[4-(1-methoxy-5,6,7,8-tetrahydro-naphthalen-2-yl)-piperidin-1-yl]-butyl}-benzamide

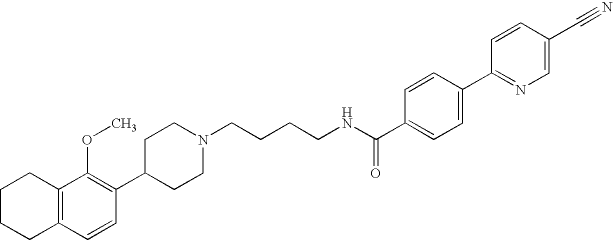

The same method was employed as in the preparation of intermediate 1 but starting from intermediate 66 and the available 2-bromo-5-cyanopyridine and gave the title compound as a beige solid in a 2.8% yield after recrystallisation from CH₃CN; m.p. 220-222° C.; LC/MS: M+H C₃₃H₃₉N₄O₂ 523.

### Example 31

### 4-(2-Cyano-thiophen-3-yl)-N-{4-[4-(1-methoxy-5,6,7,8-tetrahydro-naphthalen-2-yl)-piperidin-1-yl]-butyl}-benzamide

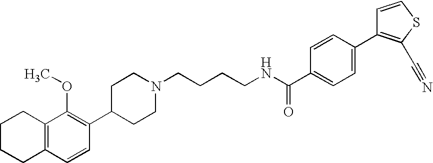

The same method was employed as in the preparation of intermediate 1 but starting from intermediate 66 and the available 3-bromo-2-cyanothiophene and gave the title compound as a brown solid in a 6.2% yield after recrystallisation from CH₃CN; m.p. 236-238° C.; LC/MS: M+H C₃₂H₃₈N₃O₂S 528.

### Example 32

### 4-(5-Chloro-pyridin-2-yl)-N-{4-[4-(1-methoxy-5,6,7,8-tetrahydro-naphthalen-2-yl)-piperidin-1-yl]-butyl}-benzamide

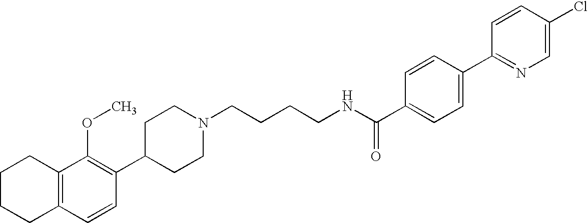

The same method was employed as in the preparation of intermediate 1 but starting from intermediate 66 and the available 2-bromo-5-chloropyridine and gave the title compound as a white solid in a 6.2% yield after recrystallisation from CH₃CN; m.p. 213-215° C.; LC/MS: M+H C₃₂H₃₉ClN₃O₂ 532.

### Example 33

### 4-(5-Acetyl-thiophen-3-yl)-N-{4-[4-(1-methoxy-5,6,7,8-tetrahydro-naphthalen-2-yl)-piperidin-1-yl]-butyl}-benzamide

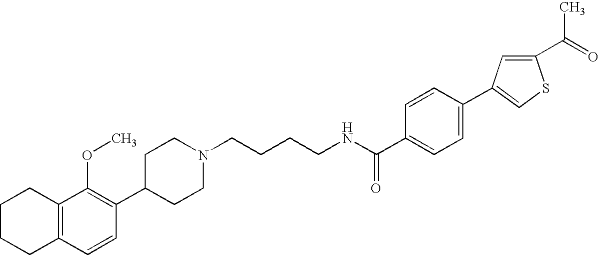

The same method was employed as in the preparation of intermediate I but starting from intermediate 66 and the available 1-(4-bromo-2-thienyl)ethan-1-one and gave the title compound as a white solid in 3.15% yield after recrystallisation from CH₃CN; m.p. 208-210° C.; LC/MS: M+H C₃₃H₄₁N₂O₃S 545.

### Example 34

### 4-(6 Chloro-pyridin-3-yl-N-{4-[4-(1-methoxy-5,6,7,8-tetrahydro-naphthalen-2-yl)-piperidin-1-yl]-butyl}-benzamide

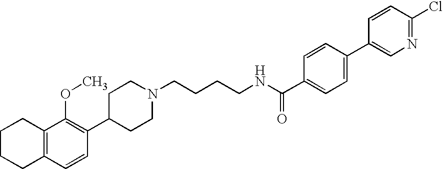

The same method was employed as in the preparation of intermediate 1 but starting from intermediate 66 and intermediate 79 and gave the title compound as a beige solid in a 30.7% yield after recrystallisation from CH₃CN; m.p. 202-204° C.; LC/MS: M+H C32H₃₉ClN₃O₂ 532.

### Example 35

### 4-(6-Chloro-pyridazin-3-yl)-N-{4-[4-(1-methoxy-5,6,7,8-tetrahydro-naphthalen-2-yl)-piperidin-1-yl]-butyl}-benzamide

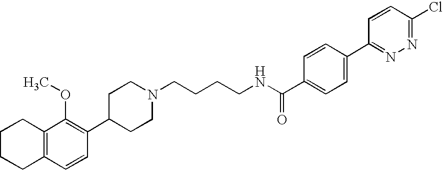

The same method was employed as in the preparation of intermediate 1 but starting from intermediate 66 and intermediate 80 and gave he title compound as a white solid in 38.7% yield after recrystallisation from CH₃CN; m.p. 192-194° C.; LC/MS: M+H C₃₁H₃₈ClN₄O₂ 533.

### Example 36

### 4-(5-Cyano-furan-2-yl-N-{4-[4-(1-methoxy-5,6,7,8-tetrahydro-naphthalen-2-yl)-piperidin-1-yl]-butyl}-benzamide

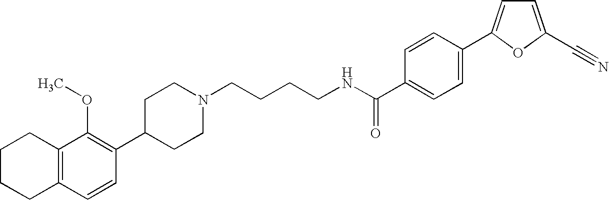

The same method was employed as in the preparation of intermediate 1 but starting from intermediate 66 and the available 5-bromo-2-furancarbonitrile and gave the title compound as a white solid in 21.7% yield after recrystallisation from CH₃CN; m.p. 180-182° C.; LC/MS: M+H C₃₂H₃₈N₃O₃ 512.

### Example 37

### 5-(4-Chloro-phenyl)-pyridine-2-carboxylic acid {4-[4-(1-methoxy-5,6,7,8-tetrahydro-naphthalen-2-yl)piperidin-1-yl]-butyl}-amide

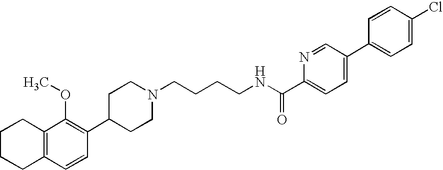

The same method was employed as in the preparation of example 1 but starting from intermediate 19 and intermediate 82 and gave the title compound as a white solid in a 9.5% yield after recrystallisation from CH₃CN; m.p. 103-105° C.; LC/MS: M+H C₃₂H₃₉ClN₃O₂ 532.

### Example 38

### 6-(4-Chloro-phenyl)-N-{4-[4-(1-methoxy-5,6,7,8-tetrahydro-naphthalen-2-yl)-piperidin-1-yl]-butyl}-nicotinamide

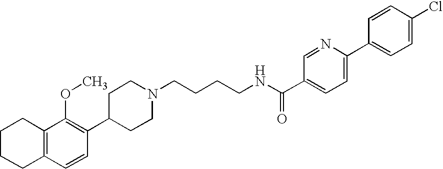

The same method was employed as in the preparation of example 1 but starting from intermediate 19 and intermediate 84 and gave the title compound as a white solid in a 47.4% yield after recrystallisation from CH₃CN; m.p. 184-186° C.; LC/MS: M+H C₃₂H₃₉ClN₃O₂ 532.

### Example 39

### 6-(4-Cyano-phenyl)-N-{4-[4-(1-methoxy-5,6,7,8-tetrahydro-naphthalen-2-yl)-piperidin-1-yl]-butyl}-nicotinamide

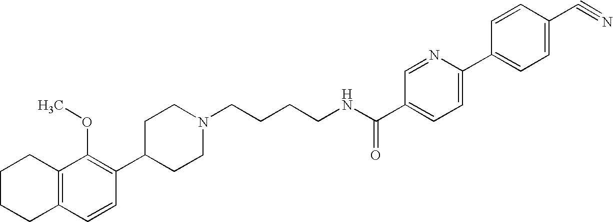

The same method was employed as in the preparation of example 1 but starting from intermediate 19 and intermediate 86 and gave the title compound as a white solid in a 21.7% yield after recrystallisation from CH₃CN; m.p. 166-168° C.; LC/MS: M+H C₃₃H₃₉N₄O₂ 523.

### Example 40

### 6-(5-Chloro-thiophen-2-yl)-N-{4-[4-(1-methoxy-5,6,7,8-tetrahydro-naphthalen-2-yl)-piperidin-1-yl]-butyl}-nicotinamide

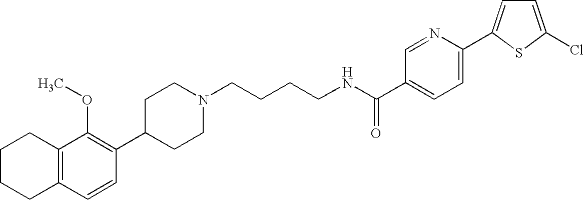

The same method was employed as in the preparation of example 1 but starting from intermediate 19 and intermediate 88 and gave the title compound as a beige solid in a 45% yield after recrystallisation from CH₃CN; m.p. 175-177° C.; LC/MS: M+H C₃₀H₃₇ClN₃O₂S 538.

### Example 41

### 5-(4-Cyano-phenyl)-pyridine-2-carboxylic acid {4-[4-(1-methoxy-5,6,7,8-tetrahydro-naphthalen-2-yl)-piperidin-1-yl]-butyl}-amide

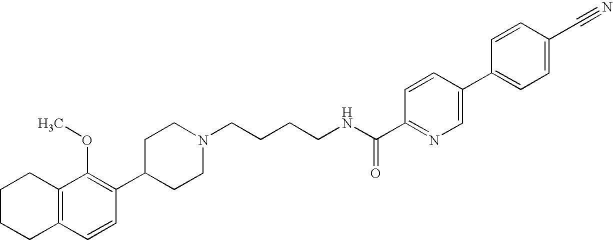

The same method was employed as in the preparation of example 1 but starting from intermediate 19 and intermediate 89 and gave the title compound as a beige solid in a 1.54% yield after recrystallisation from IprO₂; m.p. 140° C. (gummy solid); LC/MS: M+H C₃₃H₃₉N₄O₂ 523.

### Example 42

### 6-(5-Cyano-thiophen-2-yl)-N-{4-[4-(1-methoxy-5,6,7,8-tetrahydro-naphthalen-2-yl)-piperidin-1-yl]-butyl}-nicotinamide

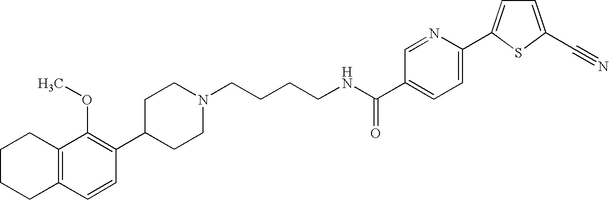

The same method was employed as in the preparation of example 1 but starting from intermediate 19 and intermediate 91.and gave the title compound as a white solid in 14.7% yield after recrystallisation: from CH₃CN; m.p.186-188° C.; LC/MS: M+H C₃₁H₃₇N₄O₂S 529.

### Example 43

### 4-(6-Cyano-pyridin-3-yl)-N-{4-[4-(1-methoxy-5,6,7,8-tetrahydro-naphthalen-2-yl)-piperidin-1-yl]-butyl}-benzamide

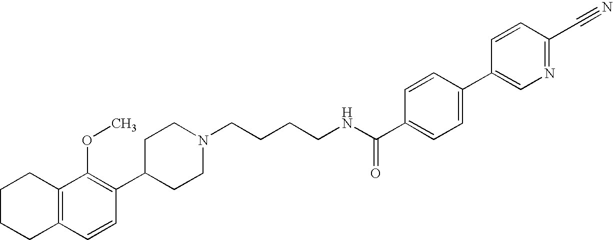

The same method was employed as in the preparation of intermediate 1 but starting from intermediate 66 and intermediate 92 and gave the title compound as a white solid in a 11% yield after recrystallisation from CH₃CN; m.p. 179-181° C.; LC/MS: M+H C₃₃H₃₉N₄O₂ 523.

### Example 44

### 4-(2-Cyano-pyrimidin-5-yl)-N-{4-[4-(1-methoxy-5,6,7,8-tetrahydro-naphthalen-2-yl)-piperidin-1-yl]-butyl}-benzamide

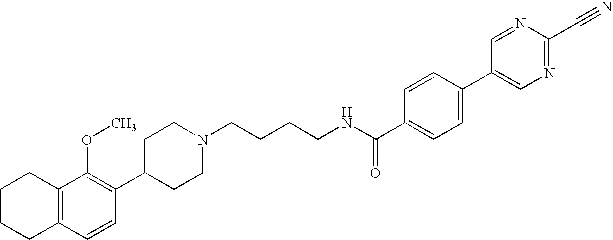

The same method was employed as in the preparation of intermediate 1 but starting from intermediate 66 and the available 5-bromo-2-pyrimidinecarbonitrile gave the title compound as a white solid in a 11% yield after recrystallisation from CH₃CN; m.p. 202° C. (gummy solid); LC/MS: M+H C₃₂H₃₈N₅O₂ 524.

### Example 45

### 2-(4-chlorophenyl)-1,4-dimethyl-1H-imidazole-5-carboxylic acid {4-[4-(1-methoxy-5,6,7,8-tetrahydro-naphthalen-2-yl)-piperidin-1-yl]-butyl}-amide

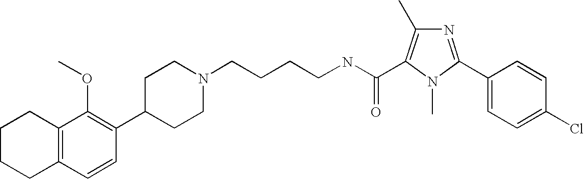

The same method was employed as in the preparation of the example 1 but starting from intermediate 19 and intermediate 96 and gave the title compound as a white solid in a 20% yield after recrystallisation from IprO₂; m.p.150° C.; LC-T of: ES+ Calculated, 549.2972; Found, 549.2996 −4.4 ppm.

Biological Assays

The Examples were tested in vivo and/or in vitro according to the following assay methods.

In Vitro Assay

HepG₂ cells, stably transfected with a construct comprising the LDL-r promoter and the luciferase reporter gene, were seeded at 50,000 cells/well in 96 well plates. After 1 day cells were incubated with compounds for 24 hours in RPMI medium containing 2% of lipoprotein-deficient serum. Compounds were tested from 10⁻⁶M to 10⁻⁹M. Cell lysates were prepared and the luciferase activity was measured by the luciferase assay system (Promega). Induction of luciferase activity was calculated taking untreated cells as control. The ED₅₀ of each compounds was determined compared to the ED₅₀ of an internal standard.

In Vivo Assay

Compounds were prepared for oral administration by milling with 0.5% hydroxypropylmethylcellulose and 5% Tween 80. Hamsters were fed for 2 weeks with a diet containing 0.2% of cholesterol and 10% of coconut oil. Then compounds were administered once a day for 3 days, from 20 to 0.2 mg/kg. Plasma lipid levels including total cholesterol, VLDL/LDL cholesterol, VLDL/LDL triglycerides and HDL-cholesterol were determined after ultracentrifugation (density 1.063 g/ml to separate VLDL/LDL fraction and HDL fraction) using the Biomerieux enzymatic kit. Reductions in VLDL/LDL cholesterol and TG plasmatic levels were calculated taking solvent treated animals as control and ED₅₀ of each compound was determined.

The compounds of the invention are potent and specific inducers of LDL-r expression.

Using the above in vitro assay all Examples of the invention induced luciferase activity having EC₅₀ values in; the range 1 nM to 300 nM.

2-Hydroxymethyl-4′-trifluoromethyl-biphenyl-4-carboxylic acid {4-[4-(1H-indol-3-yl)-piperidin-1-yl]-butyl}-amide (Example 1) had an EC₅₀ value of 7 nM.

4-(5-Chloro-pyridin-2-yl)-N-{4-[4-(1methoxy-5,6,7,8-tetrahydro-naphthalen-2-yl)-piperidin-1-yl]-butyl}-benzamide (Example 32) had an EC₅₀ value of 10 nM.

1. A compound of formula (I), and physiologically acceptable prodrugs, salts or solvates thereof; 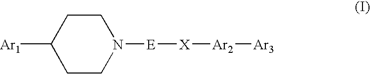
wherein
Ar₁ is:
(i) phenyl, naphthyl or phenyl fused by a C₃₋₈cycloalkyl; or
(ii) heterocyclyl selected from the group consisting of: monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein indiyldual rings of said radicals may be independently saturated, partially unsaturated or aromatic, provided that at least one ring is aromatic;
where Ar₁ is optionally substituted by 1-4 R¹ groups which may be the same or different;
Ar₂ is a phenyl group, a 5-6 membered heteroaromatic group or a bicyclic heteroaromatic group, each of which is optionally substituted by 1-4 groups independently selected from the group consisting of: C₁₋₄alkyl, halogen, hydroxy, C₁₋₄alkoxy, C₁₋₆acyl, C₁₋₆acyloxy, amino, C₁₋₄alkylamino, di-C₁₋₄alkylamino, —(CH₂)ₙOH, —(CH₂)ₙNRₓR_{y}, —O(CH₂)ₙO(CH₂)ₘOR^{a}, —O(CH₂)ₙC(O)NRₓR_{y}, —O(CH₂)ₙCN, C₂₋₅alkenyl, —O(CH₂)ₙCO₂R^{a}, —OSO₂(CH₂)ₚCH₃, —OSO₂NRₓR_{y} and —CO₂(CH₂)ₚCH₃;
Ar₃ is:
(i) phenyl, naphthyl or phenyl fused by a C₃₋₈cycloalkyl; or
(ii) heterocyclyl selected from the group consisting of monocyclic radicals and fused polycyclic radicals, wherein said radicals contain a total of from 5-14 ring atoms, wherein said radicals contain a total of from 1-4 ring heteroatoms independently selected from oxygen, nitrogen and sulfur, and wherein individual rings of said radicals may be independently saturated, partially unsaturated, or aromatic, providing that at least one ring is aromatic,
wherein Ar₃ is optionally substituted by 1-4 groups independently selected from the group consisting of: hydroxy, C₁₋₄alkyl, C₁₋₄alkoxy, C₂₋₄alkenyl, C₂₋₄alkenyloxy, C₁₋₄perfluoroalkoxy, C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, nitro, C₁₋₄perfluoroalkyl, C₁₋₄alkylcarbonyl, fluoroC₁₋₄alkylcarbonyl, C₁₋₄alkoxycarbonyl, aminocarbonyl, C₁₋₄alkylaminocarbonyl, di-C₁₋₄alkylaminocarbonyl, C₁₋₄alkylsulfonyl, C₁₋₄alkylaminosulfonyl, di-C₁₋₄alkylaminosulfonyl, C₁₋₄alkylsulfonyl and C₁₋₄alkylsulfoxy;
E is —C₁₋₆alkylene-;
X is —CONR^{a} — or —NR^{a}CO— (where the left hand side of the linkage is attached to E);
wherein
R¹ is halogen, C₁₋₄alkoxy or C₁₋₄alkyl;
R^{a} is C₁₋₄alkyl or hydrogen;
Rₓ and R_{y} are independently hydrogen, C₁₋₄alkyl, hydroxy or C₁₋₄alkoxy, where Rₓ and R_{y} are not both hydroxy or both C₁₋₄alkoxy; or Rₓ and R_{y} together with the nitrogen to which they are attached form a 5-membered ring which ring is optionally substituted by —O(CH₂)ₙC(O)NRₓR_{y}, —O(CH₂)ₙCN, —O(CH₂)ₙO(CH₂)ₘOR^{a}, —O(CH₂)ₙCO₂R^{a}, —OSO₂NRₓR_{y}, —OSO₂(CH₂)ₚCH₃, —(CH₂)ₙC(O)NRₓR_{y}, —(CH₂)ₙCN, —(CH₂)ₙO(CH₂)ₘOR^{a}, —(CH₂)ₙCO₂R^{a}, —(CH₂)ₙC(O)R^{a}, —SO₂NRₓR_{y}, —SO₂(CH₂)ₚCH₃, —CH═CHC(O)NRₓR_{y}, —CH═CHCN, —CH═CHCO₂R^{a}, —CO₂R^{a}, —C(O)R^{a}, —C(O)NRₓR_{y} and C₂₋₅alkenyl;
n and m are independently 1-4; and
p is 0-4.

2. A compound according to claim 1 wherein Ar₁ is phenyl, naphthyl, 1,2,3,4-tetrahydronaphthyl, indolyl, benzofuranyl, benzothiophenyl or indazolyl.

3. A compound according to claim 2 wherein Ar₁ is phenyl, 1,2,3,4-tetrahydronaphthyl or indolyl.

4. A compound according to claim 1 wherein E is n-butylene.

5. A compound according to claim 1 wherein X is —NR^{a}CO—.

6. A compound according to claim 1 wherein Ar₂ is phenyl, pyridyl, thiazolyl, oxazolyl, pyrazolyl or imidazolyl.

7. A compound according to claim 6 wherein Ar² is optionally substituted by one or two substituents independently selected from the group: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} (where Rₓ and R_{y} are independently hydrogen or C₁₋₄alkyl and n is 1-3) or —CO₂(CH₂)ₚCH₃ (where p is 0-3).

8. A compound according to claim 1 wherein Ar₃ is phenyl, pyridyl, pyridazinyl, pyrimidinyl, furyl or thienyl.

9. A compound according to claim 8 wherein Ar₃ is substituted by C₁₋₄alkylsulfonylamino, fluoroC₁₋₄alkylsulfonylamino, C₁₋₄alkylcarbonylamino, fluoroC₁₋₄alkylcarbonylamino, halogen, nitrile, C₁₋₄perfluoroalkyl, C₁₋₄alkylcarbonyl, fluoroC₁₋₄alkylcarbonyl, aminocarbonyl, C₁₋₄alkylaminocarbonyl or di-C₁₋₄alkylaminocarbonyl.

10. A compound according to claim 1 wherein
Ar₁ is phenyl, naphthyl, 1,2,3,4-tetrahydronaphthyl, indolyl, benzofuranyl, benzothiophenyl or indazolyl; where Ar₁ is optionally substituted by 1-4 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NR^{a}CO—;
R¹ is halogen, C₁₋₄alkoxy or C₁₋₄alkyl;
R^{a} is C₁₋₄alkyl or hydrogen;
Rₓ and R_{y} are independently hydrogen or C₁₋₄alkyl;
n is 1-3; and
p is 0-3.

11. A compound according to claim 1 wherein
Ar₁ is phenyl, 1,2,3,4-tetrahydronaphthyl or indolyl; where Ar₁ is optionally substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl;
Rₓ and R_{y} are independently hydrogen or C₁₋₄alkyl;
n is 1-3; and
p is 0-3.

12. A compound according to claim 1 wherein
Ar₁ is phenyl, 1,2,3,4-tetrahydronaphthyl or indolyl; where Ar₁ is substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: hydroxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl;
Rₓ and R_{y} are independently hydrogen or C₁₋₄alkyl;
n is 1-3; and
p is 0-3.

13. A compound according to claim 1 wherein Ar₁ is phenyl, 1,2,3,4-tetrahydronaphthyl or indolyl; where Ar₁ is optionally substituted by 1-2 R¹ groups which may be the same or different;
Ar₂ is pyridyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl;
Rₓ and R_{y} are independently hydrogen or C₁₋₄alkyl;
n is 1-3; and
p is 0-3.

14. A compound according to claim 1 wherein
Ar₁ is phenyl, 1,2,3,4-tetrahydronaphthyl or indolyl; where Ar₁ is optionally substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is phenyl, pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, halogen (such as chlorine), nitrile, C₁₋₄perfluoroalkyl, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl;
Rₓ and R_{y} are independently hydrogen or C₁₋₄alkyl;
n is 1-3; and
p is 0-3.

15. A compound according to claim 1 wherein
Ar₁ is phenyl, 1,2,3,4-tetrahydronaphthyl or indolyl; where Ar₁ is optionally substituted by 1-2 R¹ groups which may be the same or different;
Ar₂ is phenyl, pyridyl, thiazolyl, oxazolyl, pyrazolyl or imidazolyl; each of which is optionally substituted by 1-4 groups independently selected from the list: C₁₋₄alkyl, halogen, hydroxy, C₁₋₄alkoxy, hydroxyC₁₋₄alkyl, aminoC₁₋₄alkyl, mono-C₁₋₄alkylaminoC₁₋₄alkyl, di-C₁₋₄alkylaminoC₁₋₄alkyl, —O(CH₂)ₙC(O)NRₓR_{y} and —CO₂(CH₂)ₚCH₃;
Ar₃ is pyridyl, pyridazinyl, pyrimidinyl, furyl or thienyl; wherein Ar₃ is optionally substituted by 1-4 groups independently selected from the group consisting of: C₁₋₄alkylsulfonylamino (such as —NHSO₂CH₃, —NHSO₂CH(CH₃)₂), fluoroC₁₋₄alkylsulfonylamino (such as —NHSO₂CH₂CF₃), C₁₋₄alkylcarbonylamino, fluoroC₁₋₄alkylcarbonylamino, C₁₋₄alkylcarbonyl, fluoroC₁₋₄alkylcarbonyl, aminocarbonyl, C₁₋₄alkylaminocarbonyl and di-C₁₋₄alkylaminocarbonyl;
E is n-butylene;
X is —NHCO—;
R¹ is C₁₋₄alkoxy or C₁₋₄alkyl;
Rₓ and R_{y} are independently hydrogen or C₁₋₄alkyl;
n is 1-3; and
p is 0-3.

16. A compound according to claim 1 selected from the group consisting of:
2-Hydroxymethyl-4′-trifluoromethyl-biphenyl-4-carboxylic acid {4-[4-(1H-indol-3-yl)-piperidin-1-yl]-butyl}-amide;
2-(4-Cyano-phenyl)-4-hydroxymethyl-thiazole-5-carboxylic acid {4-[4-(1-methoxy-5,6,7,8-tetrahydro-naphthalen-2-yl)-piperidin-1-yl]-butyl}-amide;
2-(4-Chloro-phenyl)-4-hydroxymethyl-thiazole-5-carboxylic acid {4-[4-(1 -methoxy-5,6,7,8-tetrahydro-naphthalen-2-yl)-piperidin-1-yl]-butyl}-amide;
5-(4-Cyano-phenyl)-2-(2-hydroxy-ethyl)-2H-pyrazole-3-carboxylic acid {4-[4-(1-methoxy-5,6,7,8-tetrahydro-naphthalen-2-yl)-piperidin-1-yl]-butyl}-amide;
4-(5-Chloro-thiophen-2-yl)-N-{4-[4-(1-methoxy-5,6,7,8-tetrahydro-naphthalen-2-yl)-piperidin-1-yl]-butyl}-benzamide;
4-(5-Chloro-pyridin-2-yl)-N-{4-[4-(1-methoxy-5,6,7,8-tetrahydro-naphthalen-2-yl)-piperidin-1-yl]-butyl}-benzamide;
4-(6-Chloro-pyridin-3-yl)-N-{4-[4-(1-methoxy-5,6,7,8-tetrahydro-naphthalen-2-yl)-piperidin-1-yl]-butyl}-benzamide;
6-(4-Chloro-phenyl)-N-{4-[4-(1-methoxy-5,6,7,8-tetrahydro-naphthalen-2-yl)-piperidin-1-yl]-butyl}-nicotinamide;
6-(4-Cyano-phenyl)-N-{4-[4-(1-methoxy-5,6,7,8-tetrahydro-naphthalen-2-yl)-piperidin-1-yl]-butyl}-nicotinamide;
6-(5-Chloro-thiophen-2-yl)-N-{4-[4-(1-methoxy-5,6,7,8-tetrahydro-naphthalen-2-yl)-piperidin-1-yl]-butyl}-nicotinamide; and
2-(4-chlorophenyl)-1,4-dimethyl-1H-imidazole-5-carboxylic acid {4-[4-(1-methoxy-5,6,7,8-tetrahydro-naphthalen-2-yl)-piperidin-1-yl]-butyl}-amide.

17. A pharmaceutical composition comprising a compound as defined in claim 1 and a pharmaceutically acceptable carrier or diluent.

18. (canceled)

19. (canceled)

20. A method for the treatment of a condition resulting from elevated circulating levels of LDL-cholesterol in a mammal in need thereof, said method comprising administering a therapeutically effective amount of a compound according to claim 1.
